(19) United States
(12) Patent Application Publication
(10) Pub. No.: US 2010/0204463 A1
(43) Pub. Date: Aug. 12, 2010

(54) Preparation Of Synthetic Nucleosides via Pi-Allyl Transition Metal Complex Formation

(76) Inventors: Dennis C. Liotta, Atlanta, GA (US); Yongfeng Li, Marietta, GA (US)

Correspondence Address: KING & SPALDING, 1180 PEACHTREE STREET , NE, ATLANTA, GA, 30309-3521, US

(21) Appl. No.: 12/699,322

(22) Filed: Feb. 3, 2010

Related U.S. Application Data
(63) Continuation of application No. PCT/US2008/072478, filed on Aug. 7, 2008.
(60) Provisional application No. 60/954,449, filed on Aug. 7, 2007.

(51) Int. Cl. C07H 19/06; C07D 473/18

(52) U.S. Cl. 536/28.5; 544/276

(57) ABSTRACT

This invention provides highly regioselective and stereoselective processes for preparing synthetic nucleosides. A process for the preparation of synthetic nucleosides is provided that comprises a) preparing a bicycloamide derivative, b) reacting the bicycloamide derivative with a nucleic acid base or heterocyclic base or salt thereof in the presence of a transition metal catalyst to form a cyclopentenecarboxamide, and c) cleaving a carboxamide group from the cyclopentenecarboxamide to form the synthetic nucleoside. The processes according to the invention can be used for the synthesis of a variety of anti-viral agents, including Abacavir, Carbovir, and Entecavir, as well as derivatives thereof.

### CROSS REFERENCE TO RELATED APPLICATION

This application claims priority to U.S. Provisional Patent Application No. 60/954,449, filed Aug. 7, 2007.

The U.S. government has rights in this invention by virtue of a grant from the National Institute of Health that partially funded research leading to the invention.

### FIELD OF THE INVENTION

This invention is in the area of organic synthesis of synthetic nucleosides, including carbocylic nucleosides. This invention also is related to an efficient asymmetric approach to synthesizing synthetic nucleosides, such Abacavir, Carbovir, and Entecavir, via π-allyl transition metal complex formation.

### BACKGROUND OF THE INVENTION

Acquired immune deficiency syndrome (AIDS) has rapidly become one of the major causes of death in the world. It is estimated that over 40 million people are infected with the human immunodeficiency virus (HIV), which is the causative agent of AIDS. In 1985, 3′-azido-3′-deoxythymidine (AZT) was approved as the first synthetic nucleoside to inhibit the replication of HIV. Since then, a number of other synthetic nucleoside analogs have been proven to be effective against HIV. After cellular phosphorylation to the triphosphate form by cellular kinases, the nucleotides are incorporated into a growing strand of viral DNA and cause chain termination due to the absence of the 3′-hydroxyl group.

Carbocyclic nucleosides are structural analogs to nucleosides in which the furanose oxygen is replaced by a methylene group. Similar to native nucleosides, carbocyclic nucleosides can behave as inhibitors of the enzymes. However, because carbocyclic nucleosides lack the labile glycosidic linkage between heterocycle and sugar of native nucleosides, they are not susceptible to hydrolysis by phosphorylases or phosphotransferases.

Carbocyclic nucleosides have been the subject of extensive investigation because of the variety of biological properties displayed by these compounds. Of particular interest is the potential of carbocyclic nucleosides for use in antiviral, antitumor and anticancer chemotherapeutic applications. Perhaps the best known examples of such carbocyclic nucleosides are Carbovir and Abacavir, both of which show great promise as anti-HIV agents, and Entecavir, which has been used in the treatment of hepatitis B infection.

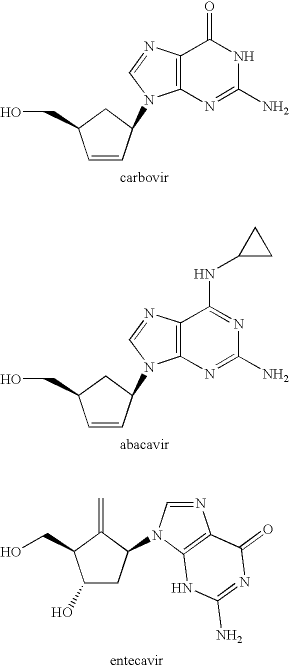

Abacavir (Ziagen; [4-(2-amino-6-cyclopropylamino-9H-purin-9-yl)-1-cyclopent-2-enyl]methanol), is a nucleoside reverse transcriptase inhibitor which has been shown to be active against HIV type 1 (HIV-1). Abacavir was approved by the Food and Drug Administration (FDA) in 1998 as nucleoside reverse transcriptase inhibitor to treat HIV-1 infection. It is thought that Abacavir is phosphorylated in vivo to its active metabolites which then compete with natural nucleosides for incorporation into viral DNA, thereby inhibiting the HIV reverse transcriptase enzyme and acting as a chain terminator of DNA synthesis. Treatment with Abacavir, alone or in combination with other anti-HIV agents decrease the viral load of greater than 99% as well as significantly improve the CD4 cell counts in patients with HIV infection, and effectiveness was maintained at least 48 weeks. Therefore, continuous improvement in the enantioselective syntheses of Abacavir nucleosides is required due to its therapeutic significance.

Carbovir (carbocyclic 2′,3′-didehydro-2′,3′-dideoxyguanosine; NSC 614846) is a potent inhibitor of HIV replication which is presumed to exert its effect by the same mechanism as other dideoxynucleosides, such as ddA, ddC or AZT, i.e. at the level of HIV reverse transcriptase (RT).

Entecavir (Baraclude; 2-amino-9-[4-hydroxy-3-(hydroxymethyl)-2-methylidene-cyclopentyl]-3H-purin-6-one) inhibits reverse transcription, DNA replication and transcription in the viral replication process.

Current synthetic routes for carbocyclic nucleosides are generally complicated and typically have low overall yields. One route to the preparation of Abacavir involves the use of γ-lactam 2-azabicyclo[2.2.1]hept-5-en-3-one (Vince lactam) as a starting material (Scheme A).

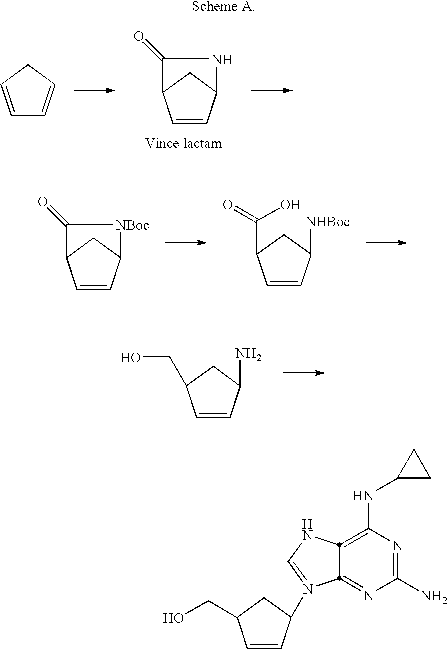

Crimmins et al. have shown a variety of methods for the synthesis of carbocyclic nucleosides, including asymmetric aldol/ring-closing metathesis (Crimmins, et al., J. Org. Chem., (2000), 65, 8499-8509) and solid phase synthesis via attachment to a polymer resin (Crimmins, et al., Org. Lett., (2000), 2(8), 1065-67).

A variety of methods for preparing this kind of carbocyclic nucleosides have been disclosed, including:

(1) Using a cycloalkene substituted with an amino group as a starting material, the desired nucleoside base is constructed on the nitrogen atom of the amino group (see J. Med. Chem., 33, 17 (1990)). However, the construction of a nucleoside structure on the N-atom requires a number of steps, which in turn increases the production cost.

(2) Directly introducing a purine structure into a 1-alkoxy-2-cyclopentene derivative in the presence of a palladium catalyst (see J. Org. Chem., 61, 4192 (1996), J. Am. Chem. Soc., 110, 621 (1988). Although this synthetic reaction requires fewer steps, it requires enantiomerically pure cyclopentene derivatives, which are difficult to manufacture.

(3) Directly introducing a purine structure into a 2-cyclopentene-1-yl-N,N-ditosylimide derivative in the presence of a palladium catalyst (see J. Org. Chem., 59, 4719 (1994), J. Org. Chem., 62, 1580 (1997)). As in the method reported above, this synthetic reaction requires enantiomerically pure cyclopentene derivatives.

While there have been many reported methods for the synthesis of various substituted carbocyclic nucleosides, particularly Abacavir, overall yields of the reported synthetic routes are generally low and the synthetic schemes require many steps. Thus, there is a need for improved methods for producing Abacavir having fewer steps and higher overall yields.

Therefore, it is an object of the present invention to provide a synthesis of carbocyclic nucleosides, including Abacavir, Carbovir and Entecavir, from inexpensive, readily available starting materials.

It is another object of the present invention to provide a synthesis of carbocyclic nucleosides, particularly Abacavir, Carbovir and Entecavir, that is efficient and does not result in the production of a significant amount of undesired isomers.

### SUMMARY OF THE INVENTION

Generally, this invention provides methods for the regioselective and stereoselective synthesis of synthetic nucleosides. As used herein, “synthetic nucleosides” refer to structural analogs of nucleosides in which the furanose oxygen is replaced by a CH₂ or C═CH₂ group.

A process for the preparation of synthetic nucleosides is provided that comprises a) preparing a bicycloamide derivative of Formula IIa or IIb, b) reacting the bicycloamide derivative of Formula IIa or IIb with a nucleic acid base, a heterocyclic base, or salt thereof in the presence of a transition metal catalyst to form a cyclopentenecarboxamide of Formula VIa or VIb, and c) cleaving a carboxamide group from the cyclopentenecarboxamide to form the synthetic nucleoside.

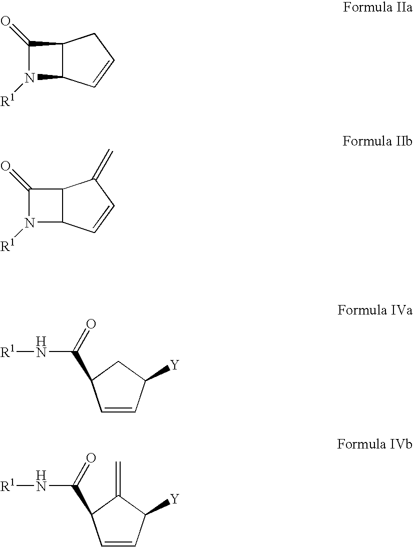

In a particular embodiment, the synthetic nucleoside is selected from the group consisted of abacavir, carbovir and entecavir. In a particular subembodiment, the synthetic nucleoside is abacavir.

In certain embodiments, the nucleic acid or heterocyclic base is a purine or pyrimidine base. In one embodiment, the nucleic acid base is a pyrimidine. In another embodiment, the nucleic acid base is a purine. In particular embodiments, the nucleic acid or heterocyclic base is a 2,6-disubstituted purine.

In one embodiment, the transition metal catalyst is optionally supported and comprises a transition metal selected from the group consisting of Ni, Fe, Co, Pd, Cu, Mo, Ru, Rh, Pt, W, and Ir. In a particular subembodiment, the transition metal catalyst comprises Pd. In one embodiment, the transition metal catalyst is supported by ligands. In one subembodiment, at least one of the ligands is a phosphine. In a particular embodiment, the transition metal catalyst is selected from the group consisting of: tetrakis(triphenylphosphine)palladium, tetrakis(triethylphosphine)palladium, tri(dibenzylideneacetone)dipalladium, bis(cycloocta-1,5-dien)palladium, di-μ-chlorobis(η-allyl)dipalladium, palladium acetate, or palladium chloride. In a particular embodiment, the transition metal catalyst is a Pd(0) or Pd(II) complex.

In certain embodiments, the above processes are provided for the preparation of synthetic nucleosides of Formula I:

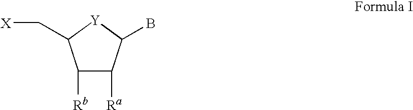

wherein Y is CH₂ or C═CH₂;
B is a purine or pyrimidine base;
X is independently H, OH, alkyl, acyl, phosphate (including monophosphate, diphosphate, triphosphate, or a stabilized phosphate prodrug), a lipid, an amino acid, a carbohydrate, a peptide or a cholesterol; and
R^{a} and R^{b} are independently selected from H, OH, alkyl, azido, cyano, alkenyl, alkynyl, Br-vinyl, —C(O)O(alkyl), —O(acyl), —O(alkyl), —O(alkenyl), Cl, Br, F, I, NO₂, NH₂, —NH(alkyl), —NH(cycloalkyl), —NH(acyl), —N(alkyl)₂, —N(acyl)₂; or R^{a} and R^{b} are taken together to form a bond.

In one embodiment, Y is CH₂. In another embodiment, Y is C═CH₂.

In one embodiment, R^{a} and R^{b} are both H. In another embodiment, R^{a} and R^{b} are not both H.

In one embodiment, R^{a} and R^{b} are taken together to form a bond. For example, when R^{a} and R^{b} are taken together to form a bond, the compound is a compound of Formula VI:

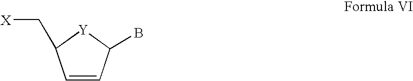

wherein Y is CH₂ or C═CH₂;
B is a purine or pyrimidine base; and
X is independently H, OH, alkyl, acyl, phosphate (including monophosphate, diphosphate, triphosphate, or a stabilized phosphate prodrug), a lipid, an amino acid, a carbohydrate, a peptide or a cholesterol.

In a particular embodiment; the synthetic nucleoside is a compound of formula VI and Y is CH₂.

In one embodiment, the synthetic nucleoside is a compound of formula I and Y is C═CH₂.

In certain embodiments, one of R^{a} and R^{b} is OH and one is H. In certain other embodiments, one of R^{a} and R^{b} is a halogen.

In certain subembodiments, one of R^{a} and R^{b} is a fluoro, and the other is selected from H and OH.

It should be noted that racemic, optically-active, or stereoisomeric forms, or mixtures thereof the synthetic nucleoside and/or its variants are also contemplated by the invention.

One aspect of the invention is to provide processes for the preparation of Abacavir [((1S,4R)-4-(2-amino-6-(cyclopropylamino)-9H-purin-9-yl)cyclopent-2-enyl)methanol], Carbovir (2-amino-9-((1R,4S)-4-(hydroxymethyl)cyclopent-2-enyl)-9H-purin-6-ol), or Entecavir [2-amino-9-[4-hydroxy-3-(hydroxymethyl)-2-methylidene-cyclopentyl]-3H-purin-6-one]. The processes utilize commercially available and inexpensive starting materials and proceed with high regioselectivity and stereochemical control. The processes represent a significant advance in the art of preparation of biologically active nucleosides, in that after formation of a novel π-allyl transition metal complex, the bicyclic precursor can be opened with complete regio- and stereo-specificity to the desired, biologically active, β-anomeric nucleoside. Non-limiting examples of the catalysts contemplated by the invention include those which contain Ni, Fe, Co, Pd, Cu, Mo, Ru, Rh, Pt, W, and Ir (e.g., see Lloyd-Jones, et al., J. Am. Chem. Soc. 2004, 126, 702-703). The transition metal catalysts optionally may be supported. In certain particularly preferred embodiments, the catalyst comprises palladium, and the palladium is used to form a π-allylpalladium complex during the synthesis.

It is believed that this is the first report of the synthesis of a nucleoside in which the regiochemistry and stereochemistry of the glycosidic linkage are controlled during this kind of bicycloamide opening. The high degree of regiocontrol and stereocontrol throughout the synthesis is highly advantageous, and may, for example, decrease the cost of production of carbocyclic nucleosides, including Abacavir, Carbovir, and Entecavir, relative to other known methods of manufacture. Further, the reagents used in the processes should be prepared easily on large scale from inexpensive materials.

Accordingly, an object of the invention is to provide a method for synthesizing Abacavir, Carbovir, or a derivative thereof. The method comprises preparing a bicycloamide derivative of Formula IIa:

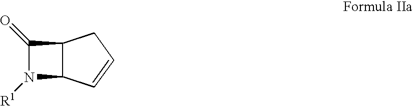

wherein R¹ is an electron-withdrawing group and then reacting the bicycloamide derivative with a nucleic acid base in the presence of a transition metal catalyst to form a cyclopentenecarboxamide. The carboxamide group of the cyclopentenecarboxamide is then cleaved to form a synthetic nucleoside, such as Abacavir, Carbovir, or a derivative thereof.

Another object of the invention is to synthesize a synthetic nucleoside, such Entecavir or a derivative thereof. The method comprises preparing a bicycloamide derivative of Formula IIb:

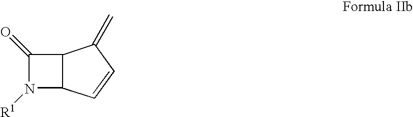

wherein R is an electron-withdrawing group. The bicycloamide derivative is then reacted with a nucleic acid base in the presence of a transition metal catalyst to form a cyclopentenecarboxamide, and the carboxamide group from the cyclopentenecarboxamide is then cleaved to form a synthetic nucleoside, for example Entecavir or a derivative thereof.

### DETAILED DESCRIPTION OF THE INVENTION

A process for the preparation of synthetic nucleosides is provided that comprises a) preparing a bicycloamide derivative of Formula IIa or IIb, b) reacting the bicycloamide derivative of Formula IIa or IIb with a nucleic acid base, a heterocyclic base, or salt thereof in the presence of a transition metal catalyst to form a cyclopentenecarboxamide, and c) cleaving a carboxamide group from the cyclopentenecarboxamide to form the synthetic nucleoside.

It has been discovered that synthetic nucleosides can be prepared regioselectively and stereoselectively by preparing a cyclopentenecarboxamide via π-allylpalladium complex formation. The cyclopentenecarboxamide compound is useful as an intermediate in the synthesis of synthetic nucleosides, for example Abacavir, Carbovir and Entecavir. Other transition metal catalysts may be used to prepare a cyclopentenecarboxamide via π-allyl transition metal complex formation.

In one embodiment, a process is provided for the preparation of intermediates for synthetic nucleosides is provided that comprises a) preparing a bicycloamide derivative of Formula IIa or IIb, b) reacting the bicycloamide derivative of Formula IIa or IIb with a nucleic acid base, a heterocyclic base, or salt thereof in the presence of a transition metal catalyst to form a cyclopentenecarboxamide.

In another embodiment, a process is provided for the preparation of a bicycloamide derivative of Formula IIa or IIb. In a subembodiment, the process of preparing a bicycloamide derivative further comprises the addition of an organolithium compound.

In another embodiment, a process is provided for the preparation of a cyclopentenecarboxamide comprising reacting the bicycloamide derivative of Formula IIa or IIb with a nucleic acid base or heterocyclic base or salt thereof in the presence of a transition metal catalyst to form a cyclopentenecarboxamide. In a subembodiment, the process further comprises cleaving a carboxamide group from the cyclopentenecarboxamide to form a synthetic nucleoside.

### Synthetic Nucleosides

A variety of synthetic nucleosides may be prepared by the processes described herein. Generally, this invention provides methods for the regioselective and stereoselective synthesis of synthetic nucleosides. As used herein, “synthetic nucleosides” refer to structural analogs of nucleosides in which the furanose oxygen is replaced by a CH₂ or C═CH₂ group.

In certain embodiments, the processes described herein are provided for the preparation of synthetic nucleosides of Formula I:

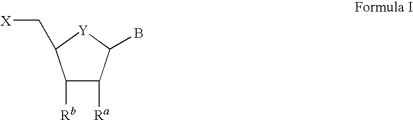

wherein Y is CH₂ or C═CH₂;
B is a purine or pyrimidine base;
X is independently H, OH, alkyl, acyl, phosphate (including monophosphate, diphosphate, triphosphate, or a stabilized phosphate prodrug), a lipid, an amino acid, a carbohydrate, a peptide or a cholesterol; and
R^{a} and R^{b} are independently selected from H, OH, alkyl, azido, cyano, alkenyl, alkynyl, Br-vinyl, —C(O)O(alkyl), —O(acyl), —O(alkyl), —O(alkenyl), Cl, Br, F, I, NO₂, NH₂, —NH(alkyl), —NH(cycloalkyl), —NH(acyl), —N(alkyl)₂, —N(acyl)₂; or R^{a} and R^{b} are taken together to form a bond.

In one embodiment, Y is CH₂. In another embodiment, Y is C═CH₂.

In one embodiment, R^{a} and R^{b} are taken together to form a bond. For example, when R^{a} and R^{b} are taken together to form a bond, the compound is a compound of Formula VI:

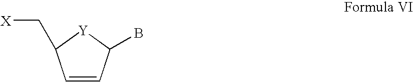

wherein Y is CH₂ or C═CH₂;
B is a purine or pyrimidine base; and
X is independently H, OH, alkyl, acyl, phosphate (including monophosphate, diphosphate, triphosphate, or a stabilized phosphate prodrug), a lipid, an amino acid, a carbohydrate, a peptide or a cholesterol.

In a particular embodiment; the synthetic nucleoside is a compound of formula VI and Y is CH₂.

In one embodiment, the synthetic nucleoside is a compound of formula I and Y is C═CH₂.

In certain embodiments, one of R^{a} and R^{b} is OH and one is H. In certain other embodiments, one of R^{a} and R^{b} is a halogen.

In certain subembodiments, one of R^{a} and R^{b} is a fluoro, and the other is selected from H and OH.

It should be noted that racemic, optically-active, or stereoisomeric forms, or mixtures thereof the synthetic nucleoside and/or its variants are also contemplated by the invention.

### Transition Metal Catalysts

A suitable catalyst is any compound or mixture of compounds that, when added to the reaction mixture, can facilitate the formation of the π-allyl transition metal complex. In one embodiment, the transition metal catalyst is optionally supported and comprises a transition metal selected from the group consisting of Ni, Fe, Co, Pd, Cu, Mo, Ru, Rh, Pt, W, and Ir. In a particular embodiment, the transition metal catalyst comprises Pd, Pt, Rh or Cu. In a particular subembodiment, the transition metal catalyst comprises Pd. In another embodiment, the catalyst comprises Cu. In another embodiment, the catalyst comprises Rh. In another embodiment, the catalyst comprises Pt.

In a particular embodiment, the transition metal catalyst or transition metal compound is selected from the group consisting of: tetrakis(triphenylphosphine)palladium, tetrakis(triethylphosphine)palladium, tri(dibenzylideneacetone)dipalladium, bis(cycloocta-1,5-dien)palladium, di-μ-chlorobis(η-allyl)dipalladium, palladium acetate, or palladium chloride. In a particular embodiment, the transition metal catalyst is a Pd(0) complex. In one embodiment, the transition metal compound is tri(dibenzylideneacetone)dipalladium, bis(cycloocta-1,5-dien)palladium, di-μ-chlorobis(η-allyl)dipalladium, palladium acetate, or palladium chloride. In another embodiment, the compound is selected from the group consisting of: tetrakis(triphenylphosphine)palladium and tetrakis(triethylphosphine)palladium.

A resin or solid supported catalysts can also be used, such as tetrakis(triphenylphosphine)palladium polymer-bound, and the like. The amount of the catalyst used in the reaction is 0.001 to 0.1 times the molar amount of bicycloamide derivative represented by the Formula IIa or IIb. The molar ratio of the transition metal catalyst to compound of Formula IIa or IIb, used in the process can be from about 0.001 to about 1, from about 0.005 to about 0.5, from about 0.008 to about 0.3, or from about 0.01 to about 0.1.

For the transition metal catalysts or transition metal compounds without phosphorous-containing ligands, the process may comprise the use of a transition metal catalysts or transition metal compounds concurrently used together with an organic phosphorus compound. Examples of the organic phosphorous compounds include aryl- or alkylphosphites such as triethylphosphite, tributylphosphite or triisopropylphosphite, are used in an amount of 1 to 10 times the molar amount of transition metal catalysts.

In one embodiment, the transition metal catalyst or transition metal compound is used without adding phosphines, phosphites or other organic phosphorus compounds. In another embodiment, the transition metal catalyst or transitional metal compound may used in the presence additional ligand compounds, such as phosphines or phosphites. For example, the both the transition metal compound and one or more ligand compounds may be added to the reaction mixture to facilitate or catalyze the formation of the π-allyl transition metal complex. Alternatively, the transition metal compound can be mixed with one more ligand compounds prior to add these compounds to the reaction mixture.

In one embodiment, the transition metal catalyst or transition metal compound is supported by ligands. A suitable ligand is any ligand that can help the metal facilitate the formation of the π-allyl transition metal complex. Suitable ligands are selected from, but not limited to, the group consisting of phosphines, for example trialkylphosphine, triarylphosphines, triphenylphosphine, tri(o-tolyl)phosphine, trifurylphosphine; bidentate phosphines, for example Ph₂P(CH₂)nPPh₂ where n=2, 3, 4, or 5; phosphites, for example tri(alkyl)phosphite, tri(aryl)phosphite, or tri(ethyl)phosphite; and arsines, for example triphenylarsine. In general, when a ligand is used in the process, the amount of ligand used is from about 1 mole percent to about 20 mole percent based on the moles of the transition metal compound.

In one embodiment a transition metal compound and one or more ligand compounds are used in the processes described herein.

In one subembodiment, at least one of the ligands is a phosphine, for example triethoxyphosphite or triphenylphosphine.

In one embodiment, the transition metal compound is selected from the group consisting of tri(dibenzylideneacetone)dipalladium, bis(cycloocta-1,5-dien)palladium, di-μ-chlorobis(η-allyl)dipalladium, palladium acetate, and palladium chloride, and is used concurrently with a organophosphorus compound, phosphine, or phosphite. In a particular embodiment, tri(dibenzylideneacetone)dipalladium or palladium acetate, is used concurrently with an aryl- or alkyl phosphite, for example triethylphosphite, tributylphosphite or triisopropylphosphite. The molar ratio of the organophosphorus compound, phosphine or phosphite compound to the transition metal compound or Pd compound used in the process is from about 1 to about 20, from about 1 to about 10, from about 1 to about 5, or from about 2 to about 5.

### Process Steps

The following examples are put forth so as to provide those of ordinary skill in the art with a complete disclosure and description of how to perform the methods and use the compositions and compounds disclosed and claimed herein. Unless indicated otherwise, parts are parts by weight, temperature is in ° C., and pressure is at or near atmospheric pressure. Standard temperature and pressure are defined as 20° C. and one atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that certain steps can be executed in a different sequence, provided that the outcome is chemically equivalent.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

The term “alkyl”, as used herein, unless otherwise specified, refers to a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, Wiley-Interscience, Third Edition, 1999, hereby incorporated by reference.

The term “acyl” refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group.

The term “aryl”, as used herein, and unless otherwise specified, refers to phenyl, biphenyl, or naphthyl, and preferably phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, Wiley-Interscience, Third Edition, 1999.

The term “purine or pyrimidine base” includes, but is not limited to, adenine, N⁶-alkylpurines, N6⁶-acylpurines (wherein acyl is C(O)(alkyl, aryl, alkylaryl, or arylalkyl)), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolo-pyrimidinyl. Purine bases include, but are not limited to, guanine, adenine, hypoxanthine, 2,6-diaminopurine, and 6-chloropurine. Pyrimidine bases include, but are not limited to, uracil, thymine, cytosine, 5-fluorocytosine, 5-methylcytosine, 6-azacytosine, 5-halouracil, 5-fluorouracil, 5-azacytosine, and 5-azauracil. Functional oxygen and nitrogen groups on the base can be protected as necessary or desired. Suitable protecting groups are well known to those skilled in the art, and include trimethylsilyl, dimethylhexylsilyl, t-butyldimethylsilyl and t-butyldiphenylsilyl, trityl, alkyl groups, and acyl groups such as acetyl and propionyl, methanesulfonyl, and p-toluenesulfonyl. Alternatively, the purine or pyrimidine base can optionally substituted such that it forms a viable prodrug, which can be cleaved in vivo. Examples of appropriate substituents include acyl moiety, an amine or cyclopropyl (e.g., 2-amino, 2,6-diamino or cyclopropyl guanosine).

The “nucleoside base” or “nucleic acid base” means a constituent base of a nucleoside as defined in the field of nucleic acid chemistry, and includes adenine, guanine, thymine, uracil, and cytosine. Additionally, the terms “nucleoside base” and “nucleic acid base” encompass purine or pyrimidine bases, as defined herein. Natural and non-natural nucleoside bases are contemplated for use in the present invention. The term “residue of nucleoside base or heterocyclic base” as used herein refers to a residual group formed by removing a hydrogen atom bonding to the nitrogen atom of the N-containing heterocyclic ring of a nucleoside base from a nucleoside base. Examples of the structures of the nucleoside bases include following purine and pyrimidine bases:

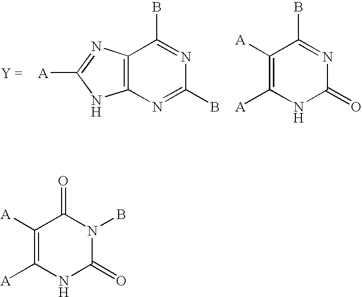

wherein A, B, and C are independently hydrogen, alkyl, halogenated alkyl, CF₃, 2-bromoethyl, alkenyl, halogenated alkenyl, bromovinyl, alkynyl, halogenated alkynyl, halo (fluoro, chloro, bromo, iodo), cyano, azido, NO₂, NH₂, —NH(alkyl), —NH(cycloalkyl), —NH(acyl), —N(alkyl)₂, —N(acyl)₂, hydroxyl, —O(acyl), —O(alkyl), —O(alkenyl), —C(O)O(alkyl), —C(O)O(alkyl); or the like.

The term “heterocyclic base” used herein refers to a series of compounds that contain a ring structure containing atoms in addition to carbon, such as sulfur, oxygen or nitrogen, as part of the ring, such as pyrrole, pyrazole; or the like.

In preferred embodiments of this method of synthesis, the process starts with two inexpensive commercially available compounds, chlorosulfonyl isocyanate and either cyclopentadiene or fulvene. The process includes, but is not limited to, [2+2] cycloaddition, kinetic resolution, tosylation and π-allylmetal formation. This process can be used to prepare a wide range of unsaturated carbocyclic nucleosides, through selection of the heterocyclic base.

The present invention also provides for a cyclopentenecarboxamide derivative and its intermediate, which is useful as an intermediate of Abacavir, Carbovir and Entecavir nucleoside synthesis. In another embodiment, the present invention relates to a highly regioselective and stereoselective method for preparing a cyclopentenecarboxamide via π-allyltransition metal complex formation. In particular embodiment, the present invention relates to a highly regioselective and stereoselective method for preparing a cyclopentenecarboxamide via π-allylpalladium complex formation.

In one embodiment, the process for the preparation of synthetic nucleosides comprises:
a) preparing a bicycloamide derivative of Formula IIa or IIb,

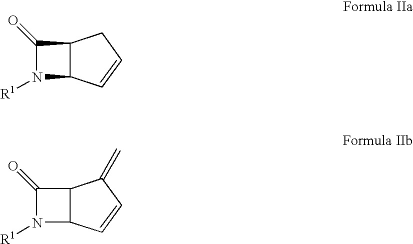

wherein each R¹ is independently an electron withdrawing group;
b) reacting the bicycloamide derivative of Formula IIa or IIb with a nucleic acid base or heterocyclic base or salt thereof in the presence of a transition metal catalyst to form a cyclopentenecarboxamide of Formula IVa or IVb;

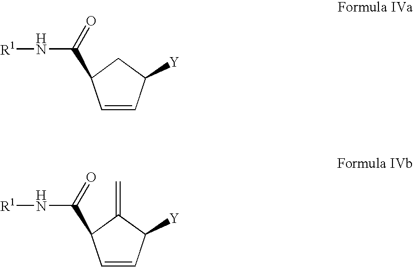

and
c) cleaving a carboxamide group from the cyclopentenecarboxamide to form the synthetic nucleoside.

In a subembodiment, the synthetic nucleoside is Abacavir, Carbovir or Entecavir.

In another subembodiment, R¹ is selected from the group consisting of benzenesulfonyl chloride, p-toluenesulfonyl chloride, p-methoxybenzenesulfonyl chloride, o-methoxybenzenesulfonyl chloride, p-nitrobenzenesulfonyl chloride, o-chlorobenzenesulfonyl chloride, p-chlorobenzenesulfonyl chloride, p-bromobenzenesulfonyl chloride, p-fluorobenzenesulfonyl chloride, 2,5-dichlorobenzenesulfonyl chloride, methylsulfonyl chloride, camphorsulfonyl chloride, chloroethanesulfonyl chloride, trifluoromethylsulfonyl chloride, and cyclohexanesulfonyl chloride.

In another subembodiment, the nucleic acid base is selected from the group consisting of adenine, N⁶-alkylpurines, N6⁶-acylpurines (wherein acyl is C(O)(alkyl, aryl, alkylaryl, or arylalkyl)), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolo-pyrimidinyl, guanine, adenine, hypoxanthine, 2,6-diaminopurine, and 6-chloropurine.

In another subembodiment, the transition metal catalyst is optionally supported and comprises a transition metal selected from the group consisting of Ni, Fe, Co, Pd, Cu, Mo, Ru, Rh, Pt, W, and Ir, for example Pd or selected from the group consisting of tetrakis(triphenylphosphine)palladium, tetrakis(triethylphosphine)palladium, tri(dibenzylideneacetone)dipalladium, bis(cycloocta-1,5-dien)palladium, di-μ-chlorobis(η-allyl)dipalladium, palladium acetate, and palladium chloride.

In one subembodiment of the process, the synthetic nucleoside is a compound of Formula I:

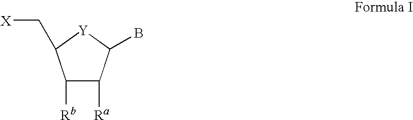

wherein Y is CH₂ or C═CH₂;
B is a purine or pyrimidine base;
X is independently H, OH, alkyl, acyl, phosphate, a lipid, an amino acid, a carbohydrate, a peptide or a cholesterol; and
R^{a} and R^{b} are independently selected from H, OH, alkyl, azido, cyano, alkenyl, alkynyl, Br-vinyl, —C(O)O(alkyl), —O(acyl), —O(alkyl), —O(alkenyl), Cl, Br, F, I, NO₂, NH₂, —NH(alkyl), —NH(cycloalkyl), —NH(acyl), —N(alkyl)₂, —N(acyl)₂; or R^{a} and R^{b} are taken together to form a bond.

In one embodiment, a process is provided for the preparation of a cyclopentenecarboxamide of Formula IVa or IVb

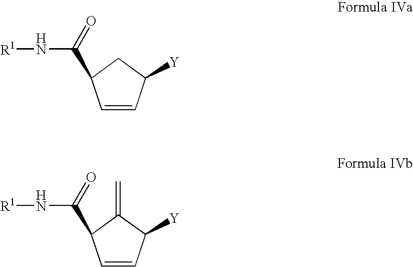

comprising:
a) preparing a bicycloamide derivative of Formula IIa or IIb,

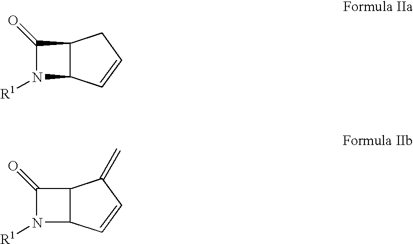

wherein each R¹ is independently an electron withdrawing group;
and
b) reacting the bicycloamide derivative of Formula IIa or IIb with a nucleic acid base or heterocyclic base or salt thereof in the presence of a transition metal catalyst to form a cyclopentenecarboxamide.

In a subembodiment, the compound of Formula IVa is selected from the group consisting of:

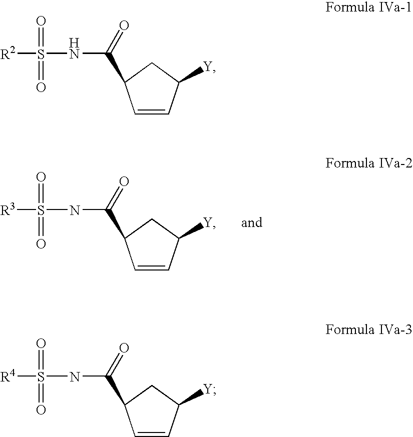

and the compound of Formula IIa is selected from the group consisting of:

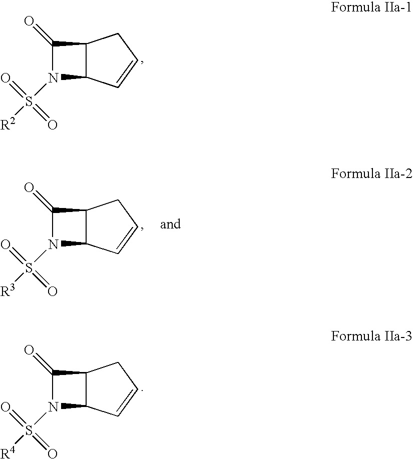

In another subembodiment, the compound of Formula IVb is selected from the group consisting of:

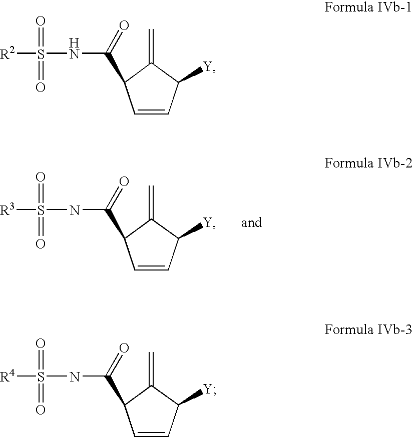

and the compound of Formula IIa is selected from the group consisting of:

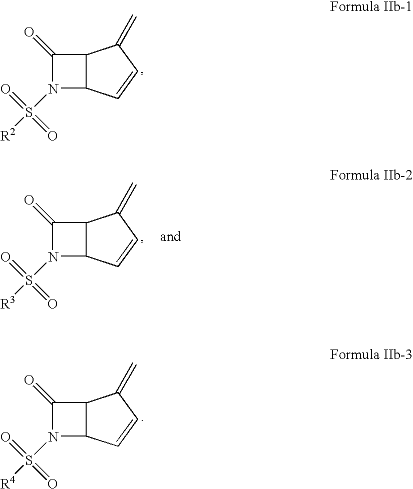

In a particular subembodiment, the transition metal catalyst comprises palladium, or is selected from the group consisting of:

tetrakis(triphenylphosphine)palladium and tetrakis(triethylphosphine)palladium, or is selected from the group consisting of: tri(dibenzylideneacetone)dipalladium, bis(cycloocta-1,5-dien)palladium, di-μ-chlorobis(η-allyl)dipalladium, palladium acetate, or palladium chloride.

In a further embodiment of the process, an organophosphorus compound is added. In a subembodiment, the organophosphorus compound is selected from the group consisting of phosphine, trialkylphosphine, triarylphosphine, triphenylphosphine, tri(o-tolyl)phosphine, trifurylphosphine, bidentate phosphine, Ph₂P(CH₂)ₙPh₂ where n=2, 3, 4, or 5; phosphite, tri(alkyl)phosphite, tri(aryl)phosphite, tri(ethyl)phosphite, arsine, and triphenylarsine. Particularly preferred are organophosphorus compounds is selected from the group consisting of phosphite, tri(alkyl)phosphite, tri(aryl)phosphite, and tri(ethyl)phosphite.

In another embodiment, a process is provided for the preparation of a bicycloamide derivative of Formula IIa or IIb

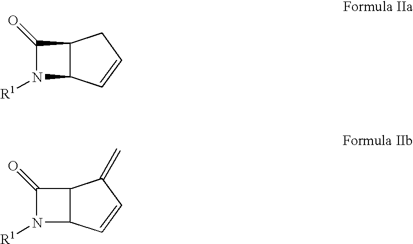

wherein each R¹ is independently an electron withdrawing group;

comprising:

reacting a compound selected from the group consisting of:

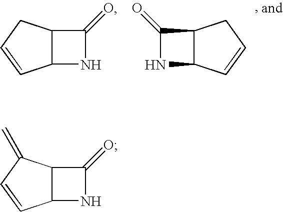

with a compound of formula III,

R¹—X Formula III

wherein X is a halogen.

In a subembodiment, the process is conducted in the presence of an organolithium compound, for example alkyl lithium compounds, methyl lithium, n-butyl lithium, t-butyl lithium, aryl lithium compounds, phenyl lithium, lithium amide bases, lithium bis(trimethylsilyl)amide, lithium diisopropylamide, or lithium 2,2,6,6-tetramethyl piperidin-1-ide.

The steps involved in the process for preparing synthetic nucleosides are outlined in detail below. The following synthetic steps are exemplary of the steps of the process for preparing a synthetic nucleoside.

### Step 1. Preparation of (1S,5R)-6-aza-bicyclo[3.2.0]hept-3-en-7-one (compound 4 in Scheme I)

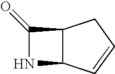

The starting materials for this process for the preparation of 6-aza-bicyclo[3.2.0]hept-3-en-7-one are cyclopentadiene and chlorosulfonyl isocyanate, as shown in Scheme I. The β-lactam is obtained in 49% yield by a [2+2] cycloaddition (see Tetrahedron Lett. 1985, 26, 1907). Further, the optically active (1S,5R)-6-aza-bicyclo[3.2.0]hept-3-en-7-one (formula I) is prepared by an easy and efficient lipase-catalyzed enantioselective ring opening of racemic β-lactam with 47% yield and 99% ee. (see Tetrahedron: Asymmetry 2004, 15, 2875)

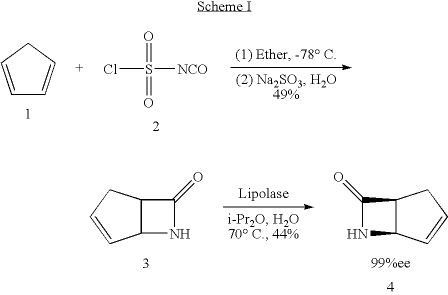

### Step 2. Preparation of corresponding bicycloamide derivative (formula IIa)

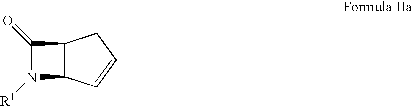

The bicycloamide derivative (formula IIa) can be obtained by reacting, in the presence of an organolithium compound and at a temperature of −78° C. to 0° C., (1S,5R)-6-aza-bicyclo[3.2.0]hept-3-en-7-one (compound 3) with a compound of formula III,

R¹—X Formula III

wherein R¹ is an electron withdrawing group, and X is a halogen atom, for example F, Cl, Br, or I. The temperature of the reaction can be or about −100° C. to about 20° C., about −100° C. to about 10° C., about −100° C. to about 5° C., about −100° C. to about 0° C., about −100° C. to about −20° C., about −100° C. to about −40° C., or about −80° C. to about −50° C. In one embodiment, R¹ is an electron withdrawing group that has at least one sulfur, phosphorus or carbon atom which will be bonded to a nitrogen atom of the amide group in the compound of Formula IIa. In certain embodiment, R¹ comprises a sulfonyl group which is bonded to the N atom of the compound of Formula IIa. In one embodiment, R¹ is substituted or unsubstituted —SO₂-alkyl or —SO₂-aryl group, for example—SO₂C₆H₄(p-CH₃), camphor sulfonyl, —SO₂C₆H₄(o-NO₂). In one embodiment, R¹ is a substituted or unsubstituted —CO₂-alkyl or —CO₂-aryl group, for example—CO₂C₆H₅. The above reaction is shown in scheme II (below).

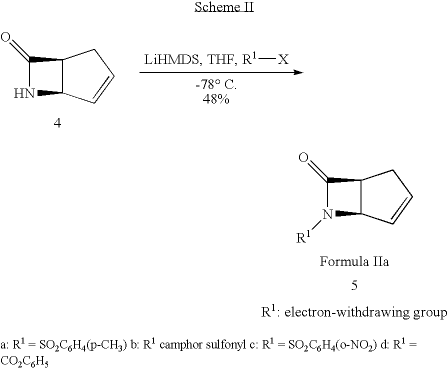

Both the bicycloamide derivative (formula IIa) and (1S,5R)-6-aza-bicyclo[3.2.0]hept-3-en-7-one (4) are somewhat unstable compounds. Thus, when the reaction to produce the bicycloamide derivative using (1S,5R)-6-aza-bicyclo[3.2.0]hept-3-en-7-one as a starting material is carried out in the presence of sodium hydride at ambient temperature as in conventional methods, it is difficult to obtain an objective bicycloamide derivative in satisfactory yields.

However, the compounds of Formula IIa can be prepared in good yield by conducting the reaction of Scheme II at a low temperature (about −78° C. to about 0° C.) in the presence of an organolithium base. The low temperature can be attained by using conventional cooling means such as liquid nitrogen or dry ice and acetone.

In one embodiment of the invention, the method of synthesis involves using an organolithium base to effectively carry out the demand reaction even at low temperatures. In one embodiment, the process of preparing a compound of Formula IIa comprises adding an organolithium base or solution of an organolithium base to the (1S,5R)-6-aza-bicyclo[3.2.0]hept-3-en-7-one compound. The organolithium base can be added to facilitate the reaction of the (1S,5R)-6-aza-bicyclo[3.2.0]hept-3-en-7-one compound with the R¹—X compound, or compound of Formula III. The organolithium bases include, for instance, alkyl lithium compounds such as methyl lithium, n-butyl lithium, and t-butyl lithium, aryl lithium compounds such as phenyl lithium, and lithium amide bases such as lithium bis(trimethylsilyl)amide, lithium diisopropylamide, and lithium 2,2,6,6-tetramethyl piperidin-1-ide and the like. The amount of the organolithium is usually 0.9 to 2 times the molar amount of (1S,5R)-6-aza-bicyclo[3.2.0]hept-3-en-7-one. The amount of the organolithium base added can be from about 0.1 to about 10, from about 0.5 to about 10, from about 0.8 to about 10, from about 0.8 to about 5, from about 0.9 to about 2 times the molar amount of (1S,5R)-6-aza-bicyclo[3.2.0]hept-3-en-7-one. This organolithium reaction is typically added slowly, for example over about 1 minute to 1 hour, about 5 minutes to 45 minutes, or about 10 to 40 minutes. Including the time to transfer the organolithium reagent, this reaction typically proceeds over the course of 5 minutes to 24 hours, or about 15 minutes to 4 hours, or about 30 minutes to 3 hours.

In one embodiment, the organolithium base is an alkyl lithium. In a particular subembodiment, the organolithium base is n-butyl lithium.

The above reaction is usually carried out in the presence of the solvent or mixture of solvents. The solvent, or mixture of solvents, includes hydrocarbons such as hexane, toluene, cyclohexane, and xylene, and ethers such as dimethoxyethane, diethyl ether, diisopropyl ether, and tetrahydrofuran. Those solvents can be used alone or in an admixture thereof. The amount of the solvent used varies depending on the type of solvent and is can be from about 0.5 to about 1000, from about 1 to about 100, or from about 10 to about 100, times the weight of the starting material.

In some embodiments, the reaction is conducted in an atmosphere of inert gas such as nitrogen or argon gas.

In one embodiment, the reaction may be carried out by supplying the compound of formula III to a reaction vessel equipped with a stirrer which is previously charged with (1S,5R)-6-aza-bicyclo[3.2.0]hept-3-en-7-one and an organolithium compound. The duration of this reaction varies depending on the reaction condition used. A suitable reaction time is from about 5 minutes to about 1 week, from about 10 minutes to about 72 hours, from about 30 minutes to about 48 hours, or from about 1 hour to about 24 hours.

Examples of compounds represented by the formula IIa are as follows. A variety of R¹ groups which have an electron withdrawing group may be used in the processes described herein. In the case where R¹ is an R—SO₂— group or R—CO₂— group, R may be a substituted or unsubstituted aromatic hydrocarbon, such as benzenesulfonyl chloride, p-toluenesulfonyl chloride, p-methoxybenzenesulfonyl chloride, o-methoxybenzenesulfonyl chloride, p-nitrobenzenesulfonyl chloride, o-chlorobenzenesulfonyl chloride, p-chlorobenzenesulfonyl chloride, p-bromobenzenesulfonyl chloride p-fluorobenzenesulfonyl chloride 2,5-dichlorobenzenesulfonyl chloride, and the like. R can also be a substituted or unsubstituted aliphatic hydrocarbon, and non-limiting examples of R¹ groups wherein R is an aliphatic hydrocarbon include methylsulfonyl chloride, camphorsulfonyl chloride, chloroethanesulfonyl chloride, trifluoromethylsulfonyl chloride, cyclohexanesulfonyl chloride, and the like. In some embodiments, R also can be a chiral aromatic or aliphatic hydrocarbon group, examples of which include (R)-(−)-10-camphorsulfonyl chloride, (R)-1-phenylpropane-1-sulfonyl chloride, (S)-1-phenylpropane-1-sulfonyl chloride, and the like.

The compound of formula III is usually used in an amount of from about 0.7 to about 10, from about 0.7 to about 5, from about 0.8 to about 3, or from about 1 to about 2, times the molar amount of (1S,5R)-6-aza-bicyclo[3.2.0]hept-3-en-7-one.

After the reaction is completed, the reaction mixture is optionally neutralized with an acid, such as acetic acid. The reaction may be subsequently added to a saturated aqueous solution of NaCl. The resulting solution is extracted with an organic solvent, for example ethyl acetate or ethyl acetate/hexanes mixture, and the solvent is distilled off from the resultant extraction. The mixture can be used without subsequent purification or may be further purified by one or more purification methods, such as column chromatography and/or recrystallization, for example crystallized from a toluene solution.

According the method of the present invention, the bicycloamide derivative represented by the formula II is obtained in a good yield, for example greater than 50%, 60%, 70%, 80%, 90% yield, or from about 50% to about 90%, from about 55% to about 90% yield, or from about 60 to 85% yield.

A variety of bicycloamide compounds of Formula IIa may be used in the processes described herein. Typical examples of the above bicycloamide derivative include the following:

(1) N-sulfonylbicycloamide derivative represented by the formula IIa-1:

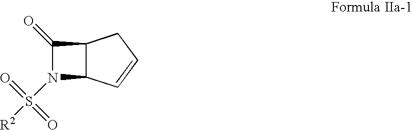

wherein R² is an optionally substituted aromatic hydrocarbon group. For example, R² may be an aryl group such as phenyl, naphthyl, anthryl, and phenanthryl; an aralkyl group such as benzyl or phenethyl, and the like. Additionally, R² may be substituted with a halogen, preferably fluorine, chlorine, bromine or iodine; a nitro group; an alkoxy group such as methoxy and ethoxy; an aralkyloxy group such as benzyloxy; an alkoxycarbonyl group such as methoxycarbonyl or ethoxycarbonyl; a cyano group; an acetyl or propionyl group; a silyloxy group such as trimethylsilyloxy or tert-butyldimethylsilyloxy; alkoxycarbonyloxy groups such as methoxycarbonyloxy or tert-butoxycarbonyloxy groups, and the like. It should be noted that in certain embodiments, R² has multiple substitutions. In a particular subembodiment, R² is a phenyl group substituted at the para-position. In certain subembodiments, R² is a phenyl group substituted at the para-position with an alkyl group, for example methyl. In other subembodiments, R² is a phenyl group substituted at the para-position with a nitro group.

(2) N-sulfonylbicycloamide derivative represented by the formula IIa-2:

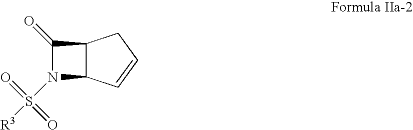

wherein R³ is a substituted or unsubstituted saturated aliphatic hydrocarbon group; non-limiting examples of which include alkyl groups, such as methyl, ethyl, tert-butyl or hexyl; cycloalkyl groups such as cyclopropyl and cyclohexyl, and the like. When R³ is substituted, the substituents may be a halogen, preferably fluorine, chlorine, bromine or iodine; a nitro group; an alkoxy group such as methoxy and ethoxy; an aralkyloxy groups such as benzyloxy; an alkoxycarbonyl group such as methoxycarbonyl or ethoxycarbonyl; cyano, acetyl or propionyl groups; silyloxy groups such as trimethylsilyloxy or tert-butyldimethylsilyloxy; alkoxycarbonyloxy groups such as methoxycarbonyloxy or tert-butoxycarbonyloxy groups, and the like. It should be noted that in certain embodiments, R³ has multiple substitutions. In certain embodiments, R³ is a halogenated alkyl group, for example trifluoromethyl.

(3) N-sulfonylbicycloamide derivative represented by the formula IIa-3:

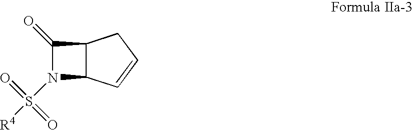

wherein R⁴ is a substituted or unsubstituted chiral hydrocarbon group. Non-limiting examples include (R)-camphor, (S)-camphor, chiral menthyl, (S)-2-phenylbutyl and the like. Those groups may be optionally substituted with a halogen, preferably fluorine, chlorine, bromine or iodine; a nitro group; an alkoxy group such as methoxy or ethoxy; aralkyloxy groups such as benzyloxy; alkoxycarbonyl groups such as methoxycarbonyl or ethoxycarbonyl; a cyano group, acetyl or propionyl; silyloxy groups such as trimethylsilyloxy or tert-butyldimethylsilyloxy; alkoxycarbonyloxy groups such as methoxycarbonyloxy or tert-butoxycarbonyloxy groups, and the like.

### Step 3. Preparation of the corresponding cyclopentenecarboxamide derivative (formula IVa)

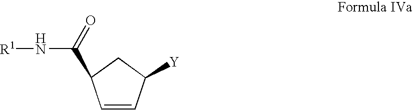

wherein R¹ is an electron withdrawing group having a sulfur, phosphorus or carbon atom directly bonded to the nitrogen atom of the amide group, and Y is a residue of a substituted or unsubstituted nucleic acid base, for example a purine or pyrimidine base, a heterocyclic base or amino, amido, azide, alkylamino, dialkylamino, arylamino, diarylamino, nitro, cyano, imine, and the like.

A method for preparing a cyclopentenecarboxamide derivative represented by the formula IV is explained below. In this reaction, shown in scheme III, the cyclopentenecarboxamide derivative (formula IVa) can be obtained by the reaction of bicycloamide derivative (formula IIa) with a nucleoside base or other bases in the presence of a transition metal catalyst, for example a palladium catalyst, in a solvent such as THF at ambient temperature.

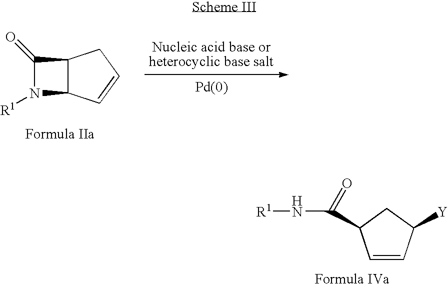

The base to form the nucleic acid or other base salt used in this reaction is not particularly limited, and includes hydrides of alkali metals, alkyl lithium or quaternary ammonium hydroxides such as tetrabutylammonium hydroxide. The amount of the base used in the reaction is 1 to 1.2 times the molar amount of the compound represented by the formula II.

The transition metal catalysts, in particular palladium catalysts, that are suitable for this method include but are not limited to tetrakis(triphenylphosphine)palladium, tetrakis(triethylphosphine)palladium, tri(dibenzylideneacetone)dipalladium, bis(cycloocta-1,5-dien)palladium, di-μ-chlorobis(η-allyl)dipalladium, palladium acetate, palladium chloride, and the like. To increase the yield of this reaction, resin or solid supported palladium catalysts can also be used, such as tetrakis(triphenylphosphine)palladium polymer-bound, and the like. The amount of the palladium catalyst used in the reaction is 0.001 to 0.1 times the molar amount of bicycloamide derivative represented by the formula II. For those palladium catalysts without phosphorous ligands, it is desired that the palladium catalyst without phosphorus ligand is concurrently used together with an organic phosphorus compound. Examples of the organic phosphorous compounds include aryl- or alkylphosphites such as triethylphosphite, tributylphosphite or triisopropylphosphite, are used in an amount of 1 to 10 times the molar amount of palladium catalysts.

The catalyst may be added as a solid or as a solution in a solvent. The organic phosphorus compound may be added as a solid, liquid or as a solution in a solvent. The transition metal compound may be added to the solution before, after or currently with the organic phosphorus compound, if an organic phosphorus compound is used. Alternatively, the transition metal compound or catalyst may be mixed with the organic phosphorus compound, optionally in a solvent, and subsequently added to the reaction mixture.

The solvent or mixture of solvents, used in the process includes, for instance, a hydrocarbon solvent such as toluene, benzene, xylenes, or hexanes; ethers such as diethyl ether, dimethoxymethane, tetrahydrofuran (THF) or dimethylsulfoxide (DMSO); nitrile such as acetonitrile; or amide such as dimethylformamide (DMF). Those solvents can be used alone or in an admixture. The amount of solvent is from about 1 to about 1000, or from about 1 to about 100, or from about 10 to about 100 times the amount by weight of the compound of formula IIa or IIb.

In certain preferred embodiments, the reaction is carried out in an atmosphere of inert gas such as nitrogen or argon gas.

The reaction may be carried out by supplying the compound represented by formula IIa to a reaction vessel equipped with a stirrer which is previously charged with nucleoside base or heterocyclic base and transition metal catalyst or palladium catalyst. The duration of this reaction is usually from 10 minutes to 24 hours, and the reaction temperature is usually between 0° C. to 100° C., or from about 0° C. to 60° C., from about 10° C. to 40° C., or from about 15° C. to 30° C.

After the reaction is completed, the reaction mixture can be concentrated and further purified by one or more purification methods, such as column chromatography and/or recrystallization.

According the method of the present invention, the cyclopentenecarboxamide derivative represented by the formula IVa is obtained in a good yield for example greater than 40%, 50%, 60%, 70%, 80%, 90% yield, or from about 45% to about 90%, from about 45% to about 65% yield, from about 55% to about 80% yield, or from about 60 to 85% yield.

A variety of bases, including purine and pyrimidine bases, can be used in the reaction to form a cyclopentencarboxamide derivative. Typical examples of the above reactions to form a cyclopentencarboxamide derivative include the following:

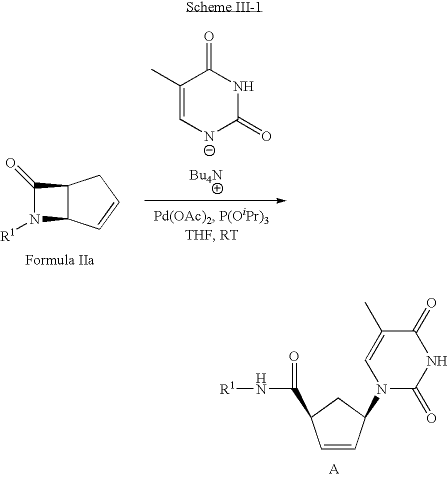

In the reaction Scheme III-1, for example, a product obtained by the present invention is compound A, where Y in the formula IVa is a thymine base.

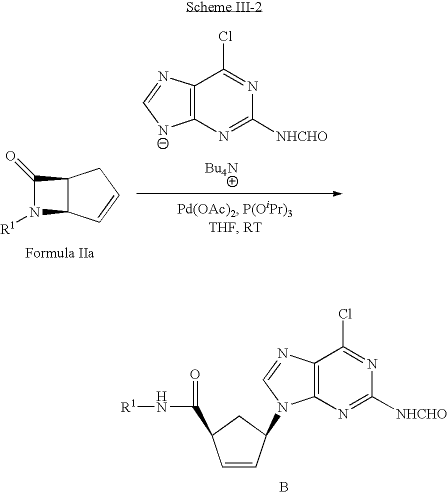

In the reaction Scheme III-2, for example, a product obtained by the present invention is compound B, where Y in the formula IVa is 2-formyl-amino-6-chloropurine-4-yl group.

Typical examples of the above cyclopentencarboxamide derivative include the following:

(1) Cyclopentenecarboxamide derivative represented by the formula IVa-1:

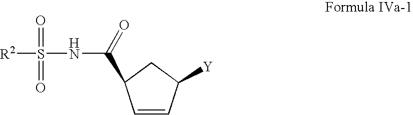

wherein R² is an aromatic hydrocarbon group which optionally may be substituted. Non-limiting examples include, for example, aryl groups such as phenyl, naphthyl. anthryl, and phenanthryl groups; aralkyl groups such as benzyl or phenethyl groups, and the like. Additionally, when R², is substituted, it may be substituted with a halogen, preferably fluorine, chlorine, bromine or iodine; a nitro group; an alkoxy groups such as methoxy or ethoxy; an aralkyloxy group such as benzyloxy; an alkoxycarbonyl group such as methoxycarbonyl or ethoxycarbonyl groups; a cyano groups, an acetyl or propionyl group; a silyloxy group such as trimethylsilyloxy or tert-butyldimethylsilyloxy; an alkoxycarbonyloxy group such as methoxycarbonyloxy or tert-butoxycarbonyloxy, and the like. Multiple substitutions of R² are also contemplated by this invention.

(2) Cyclopentenecarboxamide derivative represented by the formula IVa-2:

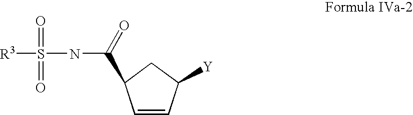

wherein R³ is a substituted or unsubstituted saturated aliphatic hydrocarbon group. Non-limiting examples include alkyl groups such as methyl, ethyl, tert-butyl or hexyl; cycloalkyl groups such as cyclopropyl and cyclohexyl groups, and the like. Those groups optionally may have a substituent such as a halogen, preferably fluorine, chlorine, bromine or iodine; a nitro group; an alkoxy group such as methoxy or ethoxy; an aralkyloxy group such as benzyloxy; an alkoxycarbonyl groups such as methoxycarbonyl, ethoxycarbonyl groups; a cyano group, an acetyl or propionyl group; a silyloxy group such as trimethylsilyloxy or tert-butyldimethylsilyloxy; an alkoxycarbonyloxy group such as methoxycarbonyloxy or tert-butoxycarbonyloxy, and the like.

(3) Cyclopentenecarboxamide derivative represented by the formula IVa-3:

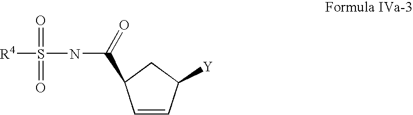

wherein R⁴ is a substituted or unsubstituted chiral hydrocarbon group, Non-limiting examples include (R)-camphor, (S)-camphor, chiral menthyl, (S)-2-phenylbuyl and the like. Those groups optionally may be substituted with a halogen, preferably fluorine, chlorine, bromine or iodine; a nitro group; an alkoxy group such as methoxy and ethoxy groups; an aralkyloxy group such as a benzyloxy group; an alkoxycarbonyl group such as methoxycarbonyl or ethoxycarbonyl; a cyano group, an acetyl or propionyl group; a silyloxy group such as trimethylsilyloxy or tert-butyldimethylsilyloxy; an alkoxycarbonyloxy groups such as methoxycarbonyloxy or tert-butoxycarbonyloxy, and the like.

The cyclopentenecarboxamide derivative is obtained as useful intermediate for synthesizing various antiviral nucleosides. Subsequent cleavage of the carboxamide group from the cyclopentenecarboxamide and optional derivatization of the nucleoside base produces the synthetic nucleoside or compound of Formula I. Such modifications are within the abilities of one of skill in the art. For example, to obtain a compound of Formula I wherein X is OH, the compound of Formula IVa is treated with a with a reducing agent and an alcohol. Alternatively, a compound of Formula I wherein X is OH may be obtained by first methylating the N of the compound of Formula IVa and then treating the compound with a reducing agent and an alcohol. In a particular embodiment, a compound of Formula IVa-1, Formula IVa-2, or Formula IVa-3 is treated with a methylating agent, such as methyl iodide or methanol, and subsequently with a reducing agent, such as NaBH₄, and an alcohol., such as methanol or ethanol.

### Synthesis of Entecavir and Entecavir Derivatives

This invention also provides for a novel synthetic route to the formation of Entecavir, the structure of which is provided below:

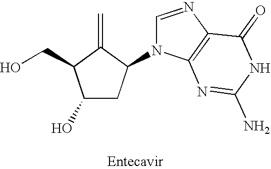

Generally, the synthesis of Entecavir can be achieved by following the reaction steps outlined above for synthetic nucleosides, including Abacavir, with the exception that fulvene is used as a starting material, rather than cyclopentadiene. An exemplary reaction scheme for the synthesis of Entecavir according to the invention is shown in Scheme IV.

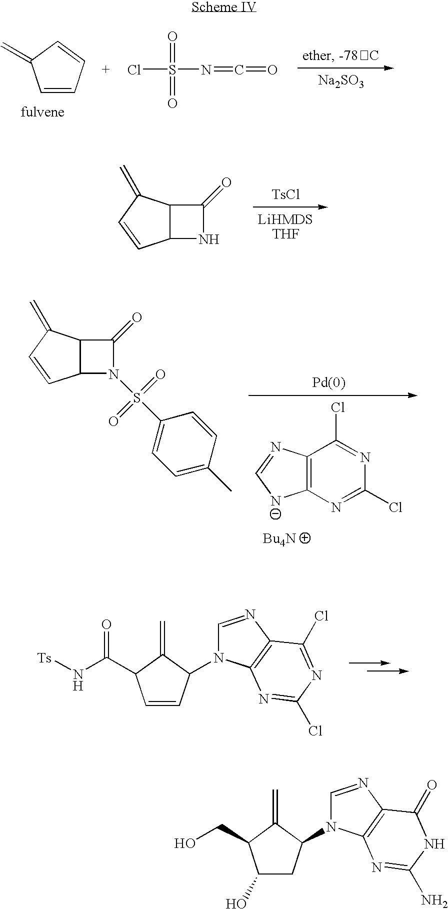

It should be noted that derivatives of Entecavir are also contemplated by the invention. In one embodiment, the processes described herein are provided for the synthesis of synthetic nucleosides of Formula I:

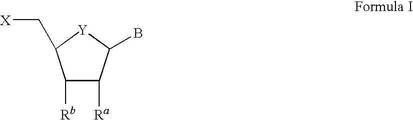

wherein Y is C═CH₂;
B is a purine or pyrimidine base, as set forth here;
X is independently H, OH, alkyl, acyl, phosphate (including monophosphate, diphosphate, triphosphate, or a stabilized phosphate prodrug), a lipid, an amino acid, a carbohydrate, a peptide or a cholesterol; and
R^{a} and R^{b} are independently selected from H, OH, alkyl, azido, cyano, alkenyl, alkynyl, Br-vinyl, —C(O)O(alkyl), —O(acyl), —O(alkyl), —O(alkenyl), Cl, Br, F, I, NO₂, NH₂, —NH(alkyl), —NH(acyl), —N(alkyl)₂, —N(acyl)₂; or R^{a} and R^{b} can come together to form a bond.

In certain embodiments, one of R^{a} and R^{b} is OH and one is H. In certain other embodiments, one of R^{a} and R^{b} is a halogen.

In certain subembodiments, one of R^{a} and R^{b} is a fluoro, and the other is selected from H and OH.

It should be noted that racemic, optically-active, or stereoisomeric forms, or mixtures thereof of Entecavir and/or its variants are also contemplated by the invention.

Analogous to the steps involved in the process for preparing synthetic nucleosides starting from cyclopentadiene, the steps for preparing synthetic nucleosides starting from fulvene are outlined in detail below. The following synthetic steps are exemplary of the steps of the process for preparing a synthetic nucleoside from fulvene.

### Step 1. Compounds of Formula IIb may be prepared by the reaction of fulvene with chlorosulfonyl isocyanate, as shown in scheme V

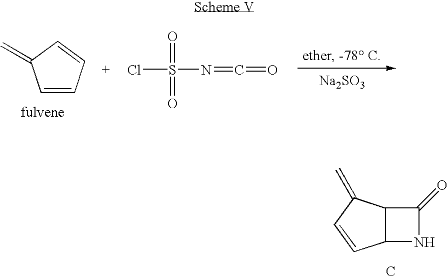

### Step 2. Preparation of corresponding bicycloamide derivative (formula IIb)

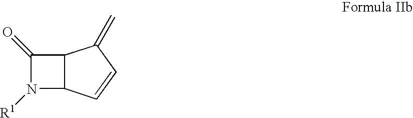

The bicycloamide derivative (formula IIb) can be obtained by reacting, in the presence of an organolithium compound and at a temperature of −78° C. to 0° C., compound C with a compound of formula III,

R¹—X (Formula III)

wherein R¹ is an electron withdrawing group, and X is a halogen atom. In one embodiment, R¹ is an electron withdrawing group that has at least one sulfur, phosphorus or carbon atom which will be bonded to a nitrogen atom of the amide group in the compound of Formula IIb. In certain embodiment, R¹ comprises a sulfonyl group which is bonded to the N atom of the compound of Formula IIb. In one embodiment, R¹ is substituted or unsubstituted —SO₂-alkyl or —SO₂-aryl group, for example—SO₂C₆H₄(p-CH₃), camphor sulfonyl, —SO₂C₆H₄(o-NO₂). In one embodiment, R¹ is a substituted or unsubstituted —CO₂-alkyl or —CO₂-aryl group, for example—CO₂C₆H₅. The above reaction is shown in scheme VI (below).

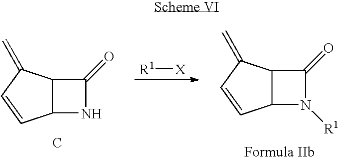

In one embodiment, compounds of Formula IIb are prepared by conducting the reaction of Scheme IV at a low temperature (−78° C. to 0° C.) in the presence of an organolithium base. The low temperature can be attained by using conventional cooling means such as liquid nitrogen or dry ice and acetone.

In one embodiment, the process of preparing a compound of Formula IIa comprises adding an organolithium base or solution of an organolithium base to compound C. The organolithium base can be added to facilitate the reaction of compound C with the R¹—X compound, or compound of Formula III. The organolithium bases include, for instance, alkyl lithium compounds such as methyl lithium, n-butyl lithium, and t-butyl lithium, aryl lithium compounds such as phenyl lithium, and lithium amide bases such as lithium bis(trimethylsilyl)amide, lithium diisopropylamide, and lithium 2,2,6,6-tetramethyl piperidin-1-ide and the like. The amount of the organolithium is usually 0.9 to 2 times the molar amount of Compound C. This organolithium reaction is typically added slowly, for example over about 1 minute to 1 hour, about 5 minutes to 45 minutes, or about 10 to 40 minutes. Including the time to transfer the organolithium reagent, this reaction typically proceeds over the course of 5 minutes to 24 hours, or about 15 minutes to 4 hours, or about 30 minutes to 3 hours.

In one embodiment, the organolithium base is an alkyl lithium. IN one embodiment, the organolithium base is lithium bis(trimethylsilyl)amide. In a particular subembodiment, the organolithium base is n-butyl lithium.

The above reaction is usually carried out in the presence of the solvent or mixture of solvents. The solvent, or mixture of solvents, includes hydrocarbons such as hexane, toluene, cyclohexane, and xylene, and ethers such as dimethoxyethane, diethyl ether, diisopropyl ether, and tetrahydrofuran. In a particular embodiment, the solvent in THF. Those solvents can be used alone or in an admixture thereof. The amount of the solvent used varies depending on the type of solvent and is usually 1 to 100 times the weight of the starting material.

In some embodiments, the reaction is conducted in an atmosphere of inert gas such as nitrogen or argon gas.

In one embodiment, the reaction may be carried out by supplying the compound of formula III to a reaction vessel equipped with a stirrer which is previously charged with Compound C and an organolithium compound. The duration of this reaction varies depending on the reaction condition used. A suitable reaction time is from about 1 hour to about 48 hours.

Examples of compounds represented by the formula IIb are as follows. In the case where R¹ is an R—SO₂— group, R may be a substituted or unsubstituted aromatic hydrocarbon, such as benzenesulfonyl chloride, p-toluenesulfonyl chloride, p-methoxybenzenesulfonyl chloride, o-methoxybenzenesulfonyl chloride, p-nitrobenzenesulfonyl chloride, o-chlorobenzenesulfonyl chloride, p-chlorobenzenesulfonyl chloride, p-bromobenzenesulfonyl chloride p-fluorobenzenesulfonyl chloride 2,5-dichlorobenzenesulfonyl chloride, and the like. R can also be a substituted or unsubstituted aliphatic hydrocarbon, and non-limiting examples of R¹ groups wherein R is an aliphatic hydrocarbon include methylsulfonyl chloride, camphorsulfonyl chloride, chloroethanesulfonyl chloride, trifluoromethylsulfonyl chloride, cyclohexanesulfonyl chloride, and the like. In some embodiments, R also can be a chiral aromatic or aliphatic hydrocarbon group, examples of which include (R)-(−)-10-camphorsulfonyl chloride, (R)-1-phenylpropane-1-sulfonyl chloride, (S)-1-phenylpropane-1-sulfonyl chloride, and the like.

The compound of formula III is usually used in an amount of 1 to 2 times the molar amount of Compound C.

After the reaction is completed, the reaction mixture is optionally neutralized with an acid, such as acetic acid. The reaction may be subsequently added to a saturated aqueous solution of NaCl. The resulting solution is extracted with an organic solvent, for example ethyl acetate or ethyl acetate/hexanes mixture, and the solvent is distilled off from the resultant extraction. The mixture can be used without subsequent purification or may be further purified by one or more purification methods, such as column chromatography and/or recrystallization, for example crystallized from a toluene solution.

According the method of the present invention, the bicycloamide derivative represented by the formula IIb is obtained in a good yield, for example greater than 50%, 60%, 70%, 80%, 90% yield, or from about 50% to about 90%, from about 55% to about 90% yield, or from about 60 to 85% yield.

A variety of bicycloamide compounds of Formula IIb may be used in the processes described herein. Typical examples of the above bicycloamide derivative include the following:

(1) N-sulfonylbicycloamide derivative represented by the formula IIb-1:

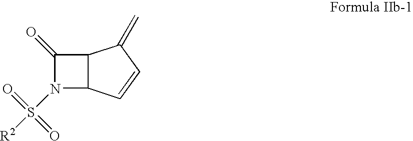

wherein R² is an substituted or unsubstituted aromatic hydrocarbon group as defined above.

(2) N-sulfonylbicycloamide derivative represented by the formula IIb-2:

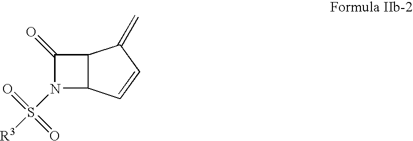

wherein R³ is a substituted or unsubstituted saturated aliphatic hydrocarbon group as defined above.

(3) N-sulfonylbicycloamide derivative represented by the formula IIb-3:

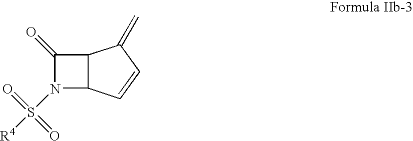

wherein R⁴ is a substituted or unsubstituted chiral hydrocarbon group as defined above.

### Step 3. Preparation of the corresponding cyclopentenecarboxamide derivative (formula IVb)

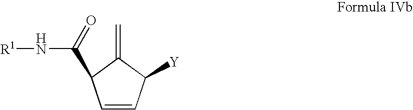

wherein R¹ is an electron withdrawing group having a sulfur, phosphorus or carbon atom directly bonded to the nitrogen atom of the amide group, and Y is a residue of a substituted or unsubstituted nucleic acid base, for example a purine or pyrimidine base, a heterocyclic base or amino, amido, azide, alkylamino, dialkylamino, arylamino, diarylamino, nitro, cyano, imine, and the like.

A method for preparing a cyclopentenecarboxamide derivative represented by the formula IVb is explained below. In this reaction, shown in scheme VII, the cyclopentenecarboxamide derivative (formula IVb) can be obtained by the reaction of bicycloamide derivative (formula IIb) with a nucleoside base or other bases in the presence of a transition metal catalyst, for example a palladium catalyst, in a solvent such as THF at ambient temperature.

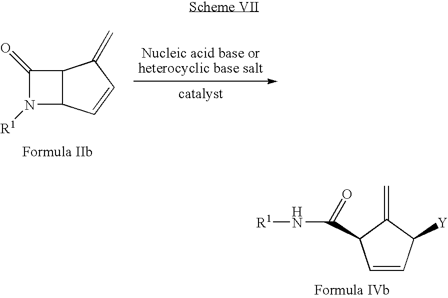

The terms “nucleoside base”, “nucleic acid base”, “heterocyclic base” and examples of nucleoside bases are as defined above. The base to form the nucleic acid or other base salt used in this reaction is not particularly limited, and includes hydrides of alkali metals, alkyl lithium or quaternary ammonium hydroxides such as tetraammonium hydroxide. The amount of the base used in the reaction is 1 to 1.2 times the molar amount of the compound represented by the formula IIb.

The transition metal catalysts for use in the reaction of compounds of Formula IIb with a nucleoside base or other bases to form the cyclopentenecarboxamide derivative (formula IVb) can be those catalysts used to prepare compounds of Formula IVa from compounds of Formula IIa and nucleoside bases or other bases. Similarly, the solvent suitable for use in this reaction are as described above.

The reaction may be carried out by supplying the compound represented by formula IIb to a reaction vessel equipped with a stirrer which is previously charged with nucleoside base or heterocyclic base and transition metal catalyst or palladium catalyst. The duration of this reaction is usually from 10 minutes to 24 hours, and the reaction temperature is usually between 0° C. to 100° C., or from about 0° C. to 60° C., from about 10° C. to 40° C., or from about 15° C. to 30° C.

After the reaction is completed, the reaction mixture is concentrated and further purified by one or more purification methods, such as column chromatography and/or recrystallization.

According the method of the present invention, the cyclopentenecarboxamide derivative represented by the formula IVb is obtained in a good yield for example greater than 40%, 50%, 60%, 70%, 80%, 90% yield, or from about 45% to about 90%, from about 45% to about 65% yield, from about 55% to about 80% yield, or from about 60 to 85% yield.

A variety of bases, including purine and pyrimidine bases, can be used in the reaction to form a cyclopentencarboxamide derivative. Typical examples of the above reactions to form a cyclopentencarboxamide derivative include the following:

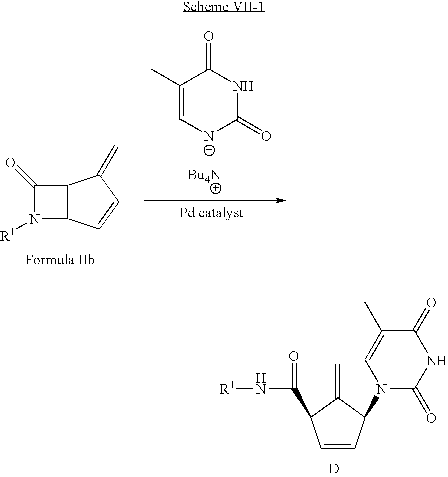

In the reaction Scheme VII-1, for example, a product obtained by the present invention is compound D, where Y in the formula IVb is a thymine base.

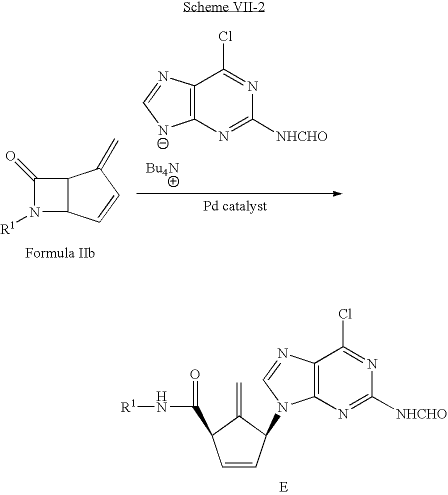

In the reaction Scheme VII-2, for example, a product obtained by the present invention is compound D, where Y in the formula IVb is 2-formyl-amino-6-chloropurine-4-yl group.

Typical examples of the above cyclopentencarboxamide derivative include the following:

(1) Cyclopentenecarboxamide derivative represented by the formula IVb-1:

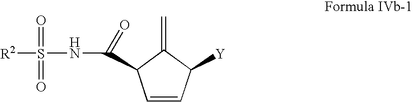

wherein R² is an aromatic hydrocarbon group which optionally may be substituted and is as defined above.

(2) Cyclopentenecarboxamide derivative represented by the formula IVb-2:

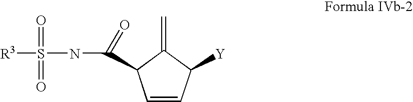

wherein R³ is a substituted or unsubstituted saturated aliphatic hydrocarbon group, and is as defined as above.

(3) Cyclopentenecarboxamide derivative represented by the formula IVb-3:

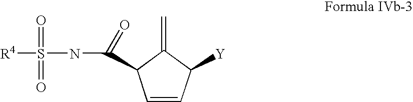

wherein R⁴ is a substituted or unsubstituted chiral hydrocarbon group and is as defined above.

The cyclopentenecarboxamide derivative is obtained as useful intermediate for synthesizing various antiviral nucleosides. Subsequent cleavage of the carboxamide group from the cyclopentenecarboxamide and optional derivatization of the nucleoside base produces the synthetic nucleoside or compound of Formula I. Such modifications are within the abilities of one of skill in the art. For example, to obtain a compound of Formula I wherein X is OH, the compound of Formula IVb is treated with a with a reducing agent and an alcohol. Alternatively, a compound of Formula I wherein X is OH may be obtained by first methylating the N of the compound of Formula IVb and then treating the compound with a reducing agent and an alcohol. In a particular embodiment, a compound of Formula IVb-1, Formula IVb-2, or Formula IVb-3 is treated with a methylating agent, such as methyl iodide or methanol, and subsequently with a reducing agent, such as NaBH₄, and an alcohol., such as methanol or ethanol.

### Stereoisomerism

Compounds of the present invention having a chiral center may exist in and be isolated in optically active and racemic forms. The present invention encompasses racemic, optically-active, or stereoisomeric form, or mixture thereof, of a compound of the invention, which possess the useful properties described herein. The optically-active forms can be prepared by, for example, resolution of the racemic form by recrystallization techniques, by synthesis from the optically-active starting materials, by chiral synthesis, or by chromatographic separation using a chiral stationary phase or by kinetic resolution, such as enzymatic resolution.

As shown below, a nucleoside contains at least two critical chiral carbon atoms (*). In general, the substituents on the chiral carbon (referred to as the 1′-substituent) and CH₂OH (referred to as the 4′-substituent) of the nucleoside can be either cis or β (on the same side) or trans or α (on the opposite sides) with respect to the sugar ring system. In this invention, the two cis enantiomers together are referred to as a racemic mixture of β-enantiomers.

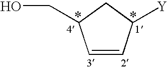

Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chrial catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
   xi) chiral gas chromatography—a technique whereby the racemate is volatilized and enantiomers are separated by virtue of their differing interactions in the gaseous mobile phase with a column containing a fixed non-racemic chiral adsorbent phase;
xi) xii) extraction with chiral solvents—a technique whereby the enantiomers are separated by virtue of preferential dissolution of one enantiomer into a particular chiral solvent;
xii) xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The processes described herein are more fully understood by reference to the following examples, which are not intended to limit the scope of the invention.

### EXAMPLES

The following examples are merely exemplary, and variations of these syntheses are also contemplated by the invention.

### Example 1

### Synthesis of Abacavir or Carbovir via (1S,5R)-6-tosyl-6-aza-bicyclo[3.2.0]hept-3-en-7-one (5a) and Pd catalyst (Scheme VIII).

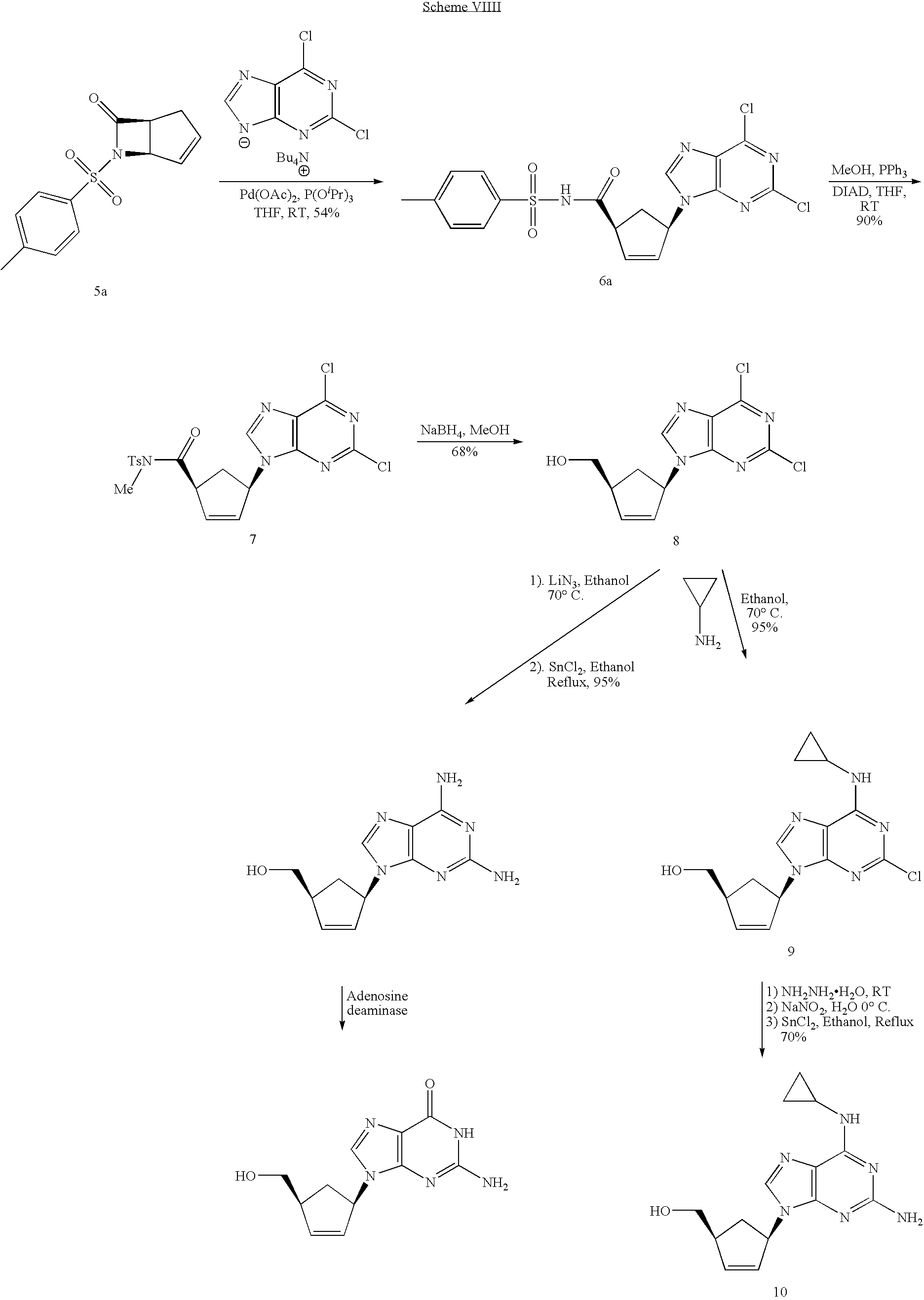

Step 1. Synthesis of (±)-6-aza-bicyclo[3.2.0]hept-3-en-7-one (3) (not shown). A solution of freshly distilled cyclopentadiene (33.0 ml, 489 mmol) in anhyd Et₂O (70 ml) was added dropwise to a solution of chlorosulfonyl isocyanate (21.3 ml) in anhydrous Et₂O (200 ml) with vigorous stirring at −78° C. After stirring at this temperature the mixture was treated at 0° C. with the solution of aq Na₂SO₃ (25%; 300 ml) and then with aqueous KOH (10%) to give pH=8. After stirring the mixture for 30 min at 0° C., the layers were separated and the aq layer was extracted with CH₂Cl₂ (3×100 ml). The combined organic layers were dried and evaporated in vacuo, and the residue purified by FC (hexane-EtOAc, 2:1) to give 3 (12.90 g, yield: 48%). ¹H NMR (400 MHz, CDCl₃): δ=2.43-2.51 (m, 1H), 2.70-2.78 (m, 1H), 3.81-3.89 (m, 1H), 4.50-4.55 (m, 1H), 5.91-5.99 (m, 1H), 6.00-6.04 (m, 1H), 6.35 (br s, 1H).

Step 2. Synthesis of (1S,5R)-6-aza-bicyclo[3.2.0]hept-3-en-7-one (4) (not shown). Crystalline racemic 3 (4 g, 36.6 mmol) was dissolved in diisopropyl ether (80 ml). Lipolase (4.0 g, 50 mg/ml) and water (0.32 ml, 17.8 mmol) were added and the mixture was shaken in an incubator shaker at 70° C. for 5 h. The reaction was stopped by filtering off the enzyme. The solvent was evaporated off and the residue (1S,5R)-6-aza-bicyclo[3.2.0]hept-3-en-7-one crystallized out. (1.8 g, yield: 45%; recrystallized from diisopropyl ether; [α]²⁵_{D}=−34.7 (C=0.45, CHCl₃); mp 76-77° C.; ee 99%).

Step 3. Synthesis of (1S,5R)-6-tosyl-6-aza-bicyclo[3.2.0]hept-3-en-7-one (5a) (not shown). A solution of 4 (2.0 g, 18.3 mmol) in anhyd THF (33 ml) was added dropwise to a stirred mixture of 1.6M n-BuLi in hexane (19.5 ml, 31.2 mmol) and anhyd THF (33 ml) at −78° C. under Ar. The mixture was stirred at −78° C. for 1 h and p-toluenesulfonyl chloride (4.65 g, 24.4 mmol) was added. The reaction temperature was raised gradually to room temperature. The solvent was evaporated in vacuo, and the residue was purified by FC (hexane-EtOAc, 4:1) to give white solid 5a (2.9 g, yield: 60%). IR (cm⁻¹) 3068, 2921, 1781, 1348, 1119; Mp: 93-95° C.; ¹H NMR (400 MHz, CDCl₃): δ=2.44 (s, 3H), 2.46-2.53 (m, 1H), 2.65-2.70 (m, 1H), 3.80-3.84 (m, 1H), 4.99-5.01 (m, 1H), 6.02 (s, 2H), 7.33 (d, 2H, J=8), 7.84 (d, 2H, J=8.4); ¹³C NMR (100 MHz, CDCl₃): δ=167.3, 145.2, 138.9, 136.5, 130.1, 128.4, 127.5, 66.2, 52.0, 31.2, 21.9; M+1: C₁₃H₁₄NO₃S 264.0688.

Step 4. Synthesis of (1S,4R)-4-(2,6-dichloro-9H-purin-9-yl)-N-tosylcyclopent-2-enecarboxamide (6a). To a stirred solution of tetrabutylammonium salt of 2,6-dichloropurine (0.67 g, 1.5 mmol) in anhydrous THF (20 ml) which was prepared from 2,6-dichloropurine and tetrabutylammonium hydroxide was dissolved in 10 ml DMF, palladium acetate (34 mg, 0.15 mmol) and triisopropyl phosphate (0.21 ml, 0.91 mmol) were added and stirred under argon at room temperature for 1 h. A solution of 5a (0.40 g, 1.5 mmol) in anhydrous THF (5 ml) was added dropwise to the resultant mixture, which was then stirred for 2 h. The solvent was evaporated in vacuo, and the residue was purified by FC(CH₂Cl₂-Methanol, 95:5) to give yellowish solid 6a (0.33 g, yield: 49%). IR (cm⁻¹) 3256, 2966, 1686, 1683, 1609, 1503; ¹H NMR (400 MHz, CDCl₃ and a drop of CD₃OD): δ=2.09-2.15 (m, 1H), 2.38 (s, 3H), 2.72-2.80 (m, 1H), 3.58-3.60 (m, 1H), 5.73-5.76 (m, 1H), 5.88-5.91 (m, 1H), 6.11-6.13 (m, 1H) 7.27 (d, 2H, J=8), 7.85 (d, 2H, J=8), 8.24 (s, 1H); ¹³C NMR (100 MHz, CDCl₃): δ=21.9, 33.8, 51.4, 59.5, 128.4, 129.8, 130.8, 131.4, 135.7, 135.7, 145.4, 145.5, 151.8, 152.8, 170.9; M+1: C₁₈H₁₆Cl₂N₅O₃S 452.0342.

Step 5. Synthesis of (1S,4R)-4-(2,6-dichloro-9H-purin-9-yl)-N-methyl-N-tosylcyclopent-2-enecarboxamide (7). To a solution of 6a (0.65 g, 1.44 mmol) in THF-CH₂Cl₂(1:1) was added methanol (0.2 ml), PPh₃ (1.33 g, 5.76 mmol), and diisopropyl azodicarboxylate (0.98 ml, 5.74 mmol) under argon atmosphere with stirring at room temperature, after being stirred for 30 mins, the solvent was evaporated in vacuo to give a residue, which was submitted to column chromatography. Elution with hexane-EtOAc (1:2) gave 0.63 g of product 7. IR (cm⁻¹) 3256, 2966, 1686, 1683, 1609, 1503; ¹H NMR (400 MHz, CDCl₃): δ=2.12-2.16 (m, 1H), 2.45 (s, 3H), 2.82-2.90 (m, 1H), 3.25 (s, 3H), 4.54-4.56 (m, 1H), 5.80-5.83 (m, 1H), 5.96-5.99 (m, 1H), 6.16-6.18 (m, 1H) 7.36 (d, 2H, J=8), 7.73 (d, 2H, J=8), 8.31 (s, 1H); ¹³C NMR (100 MHz, CDCl₃): δ=21.9, 33.7, 35.8, 51.2, 59.6, 127.5, 130.5, 130.8, 131.1, 135.9, 137.1, 145.5, 145.8, 151.8, 152.9, 153.0, 173.9; M+1: C₁₉H₁₈Cl₂N₅O₃S 466.0501.

Step 6. Synthesis of ((1S,4R)-4-(2,6-dichloro-9H-purin-9-yl)cyclopent-2-enyl)methanol (8). To a solution of 7 (0.63 g, 1.35 mmol) in methanol was added NaBH₄ (51.3 mg, 1.35 mmol) portionwise with stirring at −20° C. During this period, the internal temperature was kept below 0° C. The mixture was then stirred at room temperature for 5 h. After the reaction was neutralized with AcOH, the solvent was evaporated off in vacuo. To the residue was added water. The mixture was extracted with EtOAc. The extract was dried over MgSO₄ and condensed in vacuo to give a residue, which was purified on silica gel column chromatography. Elution with CH₂Cl₂-MeOH (95:5) afforded 0.29 g of product 8. IR (cm⁻¹) 3256, 2966, 1686, 1683, 1609, 1503; ¹H NMR (400 MHz, CDCl₃): δ=1.85-1.91 (m, 1H), 2.53 (s, 1H), 2.84-2.92 (m, 1H), 3.10-3.11 (m, 1H), 3.72 (m, 1H), 3.88 (m, 1H), 5.75-5.80 (m, 1H), 5.84-5.87 (m, 1H), 6.21-6.24 (m, 1H), 8.42 (s, 1H); ¹³C NMR (100 MHz, CDCl₃): δ=34.1, 47.7, 60.8, 64.4, 129.2, 131.1, 140.3, 145.7, 151.6, 152.8, 152.9; M+1: C₁₁H₁₁Cl₂N₄O₁ 285.0304.

Step 7. Synthesis of ((1S,4R)-4-(2-amino-6-(cyclopropylamino)-9H-purin-9-yl)cyclopent-2-enyl)methanol (10). To a solution of 8 (200 mg, 0.70 mmol) in ethanol was added cyclopropylamine (0.14 ml, 2.1 mmol), the mixture was then heated at reflux for 5 h. After evaporating the solvent, the crude product 9 was used to next reaction without further purification. The crude 11 was dissolved in hydrazine monohydrate (10 ml) and MeOH (5 ml). After heating at 50° C. for overnight, the solution was concentrated to dry and coevaporated with 2-propano (2×30 ml) until a white gum was obtained. The residue was dissolved in a 10% aqueous acetic acid solution (10 ml) and cooled in an ice bath. Sodium nitrite (0.075 g, 1.1 mmol) was added, and the mixture was stirred for 1 h. After evaporating the solvent, the crude product was dissolved in ethanol and Tin (II) chloride dehydrate (315 mg, 1.4 mmol) was added. After heating at reflux for 2 h, the mixture was cooled and evaporated. The residue was purified on silica gel column chromatography. Elution with CH₂Cl₂-MeOH (95:5) afforded 133 mg of product 10. IR (cm⁻¹) 3321, 3207, 1589, 1474; ¹H NMR (400 MHz, CDCl₃): δ=0.54-0.64 (m, 4H), 1.51-1.58 (m, 1H), 2.52-2.60 (m, 1H), 2.83 (m, 1H), 3.00 (br, 1H), 3.40-3.42 (m, 2H), 4.73 (m, 1H), 5.37 (m, 1H), 5.83 (m, 2H), 6.07 (m, 1H), 7.57 (s, 1H); ¹³C NMR (100 MHz, CDCl₃): δ=7.1, 22.0, 35.0, 48.4, 58.8, 64.8, 114.2, 130.7, 135.5, 138.7, 156.6, 160.7; M+1: C₁₄H₁₉N₆O₁ 287.1611.

Alternative Step 7. Synthesis of Carbovir. Carbovir may also be prepared from 8. As shown in Scheme VIIII, the chlorines can be replaced with amines via treatment with LiN₃ in ethanol (70° C.), followed by reaction with SnCl₂ in ethanol (reflux). (95% yield). This 2,6-diaminopurine product an be treated with adenosine deaminase to produce Carbovir.

### Example 2

### Synthesis of Abacavir via (1S,5R)-6-(p-Nitrobenzenesulfonyl)-6-aza-bicyclo[3.2.0]hep-3en-7-one 4 and Pd catalyst (Scheme X)

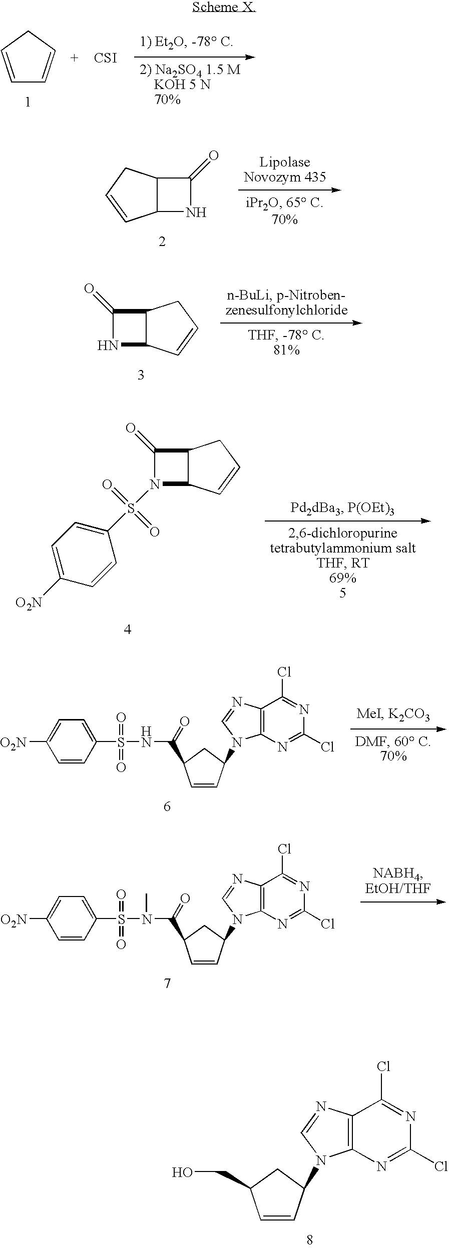

Step 1. Thermolysis of cyclopentadiene dimer 1. 200 mL of dicyclopentadiene were placed in a 250 mL flask equipped with a distillation condenser apparatus. The head of the condenser was fitted with a thermometer. The fractional distillation was performed at 165° C., and the cyclopentadiene distills smoothly at 38-46° C. After two-third of the cyclopentadiene has been pyrolyzed during the course of 5-6 hours, a higher temperature for the pyrolysis is necessary to obtain a rapid distillation. The fresh distilled cyclopentadiene 1 was trapped in a 250 mL flask immersed in a dry Ice bath. The pyrolysis was carried out under nitrogen atmosphere. ¹H NMR (400 MHz, CDCl₃): δ=3 (s, 2H), 6.5 (s, 2H), 6.8 (s, 2H).

Step 2. (±)-6-aza-bicyclo[3.2.0]hep-3en-7-one 2. A 5 L, 3-neck flask with a nitrogen/thermometer inlet, was charged with 3 L of dry diethyl ether and cooled to −78° C. The cyclopentadiene (30 g, 37.5 mL, 0.453 mole) was added to the solution. The concentration of cyclopentadiene in the reaction was 0.15 M. Chlorosulfonyl isocyanate (CSI) (61.17 g, 37.62 mL, 0.43 mole) was added drop wise to the reaction. The reaction was followed by ¹H NMR by taking an aliquot of the reaction and dissolving it in CDCL₃ to prepare an NMR sample. After 5 hours, 0.2 equivalent of chlorosulfonyl isocyanate was added, and the reaction mixture was stirred overnight. The reaction mixture was warmed to −20° C. and treated with 60 mL of 1.5 M solution of Na₂SO₃, added slowly by addition funnel. The temperature elevated to 5° C., then, treated with a cold solution of 5 N KOH 400 mL to give pH 8. The reaction mixture was allowed to warm to ambient temperature, during this process the dicyclopentadiene became insoluble. The mixture was filtered through celite to remove the cyclopentadiene dimer, and washed with 200 mL ether. The layers were separated and the organic layer was dried over magnesium sulfate and concentrated to give the lactam 2 (33 g, 70% yield) as light brown color oil. ¹H NMR (400 MHz, CDCl₃): δ=2.4-2.5 (m, 1H), 2.65-2.7 (m, 1H), 3.8 (m, 1H), 4.5 (s, 1H), 5.95 (s, 1H), 6.0 (s, 1H), 6.4 (br s, 1H).

Step 3. (1S,5R)-6-aza-bicyclo[3.2.0]hep-3en-7-one 3. In a 3-neck 3 L flask, the racemic lactam (63 g, 0.578 mole) was dissolved in 1.6 L of diisopropyl ether to produce a lactam concentration of 0.36 M. Deionized water (10.9 mL, 0.607 mole, 1.05 equiv.) was added. The solution slowly became clear when heated to 40° C. The Lipolase enzyme, Novozym 435 (31.5 g) was added to the reaction mixture and the temperature was increased to 65° C. The progress of the reaction was monitored by ¹H NMR. The amino acid formed during the enzymatic resolution was not soluble, and the formation can be observed during the reaction. The reaction time was 24 hours. After the reaction cooled to ambient temperature, the mixture was filter to recover the enzyme, and washed with 1 L of diisopropyl ether. The layers were separated and the ether phase was concentrated to afford a pale yellow solid. The material was aged in diisopropyl ether 100 mL, filtered, washed with cold diisopropyl ether solution 70 mL to give a white crystalline material 3 (22 g, 70% yield).

Step 4. (1S,5R)-6-(p-Nitrobenzenesulfonyl)-6-aza-bicyclo[3.2.0]hep-3en-7-one 4. To a solution of (1S,5R)-6-aza-bicyclo[3.2.0]hep-3en-7-one 3 (13.8 g, 0.126 mole) in dry THF (300 mL) at −78° C. was added BuLi-n-hexane solution 1.6 M (94.5 mL, 0.151 mole) over 35 minutes. The mixture was stirred for 1:30 hour then transferred by cannula to a cold −78° C. solution of p-nitrobenzenesulfonyl chloride (29.46 g, 0.133 mole) over 45 minutes. The reaction was over after the transfer was completed. The reaction was quenched with 0.2 equivalents of acetic acid (1.44 mL) and poured in 400 mL of NaCl saturated solution. The mixture was extracted with 1.5 L of ethyl acetate, concentrated down and coevaporated with toluene two times (200 mL). The crude material 41 g was crystallized in methanol to afford a pale yellow solid 4 (30 g, 81% yield). ¹H NMR (400 MHz, CDCl₃): δ=2.55 (m, 1H), 2.7 (m, 1H), 3.9 (m, 1H), 5.1 (s, 1H), 6.05 (m, 2H), 8.05 (d, 2H), 8.4 (d, 2H).

Step 5. 2,6-dichloropurine tetrabutylammonium 1:1 salt formation 5. In a 1 L flask, 2,6-dichloropurine (21.28 g, 0.112 mole) was partially dissolved in 50 mL of THF. A freshly-prepared aqueous solution of tetrabutylammonium (90 g, 0.112 mole) in 300 mL of deionized water was added to the mixture. The mixture became soluble and after 2 hours the mixture was concentrated, and coevaporated with toluene (200 mL) two times. Diethyl ether (400 mL) was poured into the gummy residue. The mixture was stirred and triturated until the base-salt product became a white solid. The ether was removed under vacuum and the product dried under high vacuum to afford 47 g of material 5. ¹H NMR (400 MHz, CDCl₃): δ=1 (m, 12H), 1.35 (m, 8H), 1.55 (m, 8H), 3.1 (m, 8H), 8.1 (s, 1H).

Step 6. (1S,4R)-4-(2,6-dichloro-9H-purin-9-yl)-N-p-Nitrobenzenesulfonylcyclopent-2-enecarboxamide 6. To a 1 L flask, charged with 500 mL of dry THF, degassed 3 times, was added Pd₂dBa₃ (3.86 g, 3.74 mmol, 0.05 equivalent) and P(OEt)₃ (3.86 mL, 11.22 mmol, 0.15 equivalent). After 30 minutes, 2,6-dichloropurine tetrabutylammonium salt 5 (32.77 g, 78.56 mmol, 1.05 equivalent) and protected lactam 4 (22 g, 74.8 mmol, 1 equivalent) were added the reaction vessel. After 50 minutes the reaction was completed. Then ⅔ of solvent were removed, 500 mL of ethyl acetate added and the organic layer was washed 3 times with 250 mL of HCl 1N solution to remove the tetrabutylammonium component. The organic layer was filtered through celite, concentrated, and coevaporated twice with toluene (100 mL). The product was crystallized in methanol to afford a pale grey solid 6 (25 g, 69% yield) The compound was a chloride salt form of the purine base, the material was washed with a NaHCO₃ saturated solution and crystallized in ethyl acetate/hexanes. ¹H NMR (400 MHz, CDCl₃ with CD₃OD): δ=2.0 (dt, 1H), 2.7 (m, 1H), 3.55 (m, 1H), 5.7 (m, 1H), 5.9 (m, 1H), 6.1 (m, 1H), 8.15 (d, 2H), 8.2 (s, 1H purine base), 8.3 (d, 2H) salt form 8.5 (s, 1H) free base.

Step 7. (1S,4R)-4-(2,6-dichloro-9H-purin-9-yl)-N-methyl-N-(4-nitrophenylsulfonyl)cyclopent-2-enecarboxamide 7. To a solution of (1S,4R)-4-(2,6-dichloro-9H-purin-9-yl)-N-p-nitrobenzenesulfonylcyclopent-2-enecarboxamide 6 (4.82 g, 0.01 mole) in dry dimethylformamide (DMF) (50 mL) was added K₂CO₃ (4.20 g, 0.03 mole) and methyl iodide (MeI) (6.6 mL, 0.1 mol). The mixture was stirred for 1 hour at 60° C. The mixture was poured in 400 mL of NaCl saturated solution. The mixture was extracted with 1.0 L of ethyl acetate, concentrated, and coevaporated with toluene two times (200 mL). The crude material was crystallized in methanol to afford a pale yellow solid 7 (3.5 g, 70% yield). ¹H NMR (400 MHz, CDCl₃): δ=2.12-2.16 (m, 1H), 2.45 (s, 3H), 2.82-2.90 (m, 1H), 3.25 (s, 3H), 4.54-4.56 (m, 1H), 5.80-5.83 (m, 1H), 5.96-5.99 (m, 1H), 6.16-6.18 (m, 1H) 7.36 (d, 2H, J=8), 7.73 (d, 2H, J=8), 8.31 (s, 1H).

Step 8. ((1S,4R)-4-(2,6-dichloro-9H-purin-9-yl)cyclopent-2-enyl)methanol 8. To a solution of (1S,4R)-4-(2,6-dichloro-9H-purin-9-yl)-N-methyl-N-(4-nitrophenyl sulfonyl)cyclopent-2-enecarboxamide 7 (0.63 g, 1.35 mmol) in 30 mL (1:1) ethanol and THF was added NaBH₄ (51.3 mg, 1.35 mmol). The mixture was then stirred at room temperature for 1 hour. After the reaction was neutralized with acetic acid, the solvent was evaporated in vacuo. To the residue was added water. The mixture was extracted with ethyl acetate. The extract was dried over MgSO₄ and condensed in vacuo to give a residue, which was purified by silica gel column chromatography. Elution with CH₂Cl₂-MeOH (95:5) afforded 0.29 g of product 8. ¹H NMR (400 MHz, CDCl₃): δ=1.85-1.91 (m, 1H), 2.53 (s, 1H), 2.84-2.92 (m, 1H), 3.10-3.11 (m, 1H), 3.72 (m, 1H), 3.88 (m, 1H), 5.75-5.80 (m, 1H), 5.84-5.87 (m, 1H), 6.21-6.24 (m, 1H), 8.42 (s, 1H).

Step 9. ((1S,4R)-4-(2-amino-6-(cyclopropylamino)-9H-purin-9-yl)cyclopent-2-enyl)methanol (Abacavir). To a solution of ((1S,4R)-4-(2,6-dichloro-9H-purin-9-yl)cyclopent-2-enyl)methanol 8 (200 mg, 0.70 mmol) in ethanol was added cyclopropylamine (0.14 mL, 2.1 mmol), the mixture was then heated at reflux for 5 h. After evaporating the solvent, the crude product was used to next reaction without further purification. The crude product was dissolved in hydrazine monohydrate (10 mL) and MeOH (5 mL). After heating at 50° C. for overnight, the solution was concentrated to dry and coevaporated with 2-propanol (2×30 mL) until a white gum was obtained. The residue was dissolved in a 10% aqueous acetic acid solution (10 mL) and cooled in an ice bath. Sodium nitrite (0.075 g, 1.1 mmol) was added, and the mixture was stirred for 1 h. After evaporating the solvent, the crude product was dissolved in dioxane, triphenyl phosphine (366 mg, 1.4 mmol) and 2 mL of ammonia hydroxide were added. After heating at reflux for 5 h, the mixture was cooled and evaporated. The residue was purified on silica gel column chromatography. Elution with CH₂Cl₂-MeOH (95:5) afforded 133 mg of final product. ¹H NMR (400 MHz, CDCl₃): δ=0.54-0.64 (m, 4H), 1.51-1.58 (m, 1H), 2.52-2.60 (m, 1H), 2.83 (m, 1H), 3.00 (br, 1H), 3.40-3.42 (m, 2H), 4.73 (m, 1H), 5.37 (m, 1H), 5.83 (m, 2H), 6.07 (m, 1H), 7.57 (s, 1H).

### Example 3

### Synthesis of Entecavir via (Scheme VI)

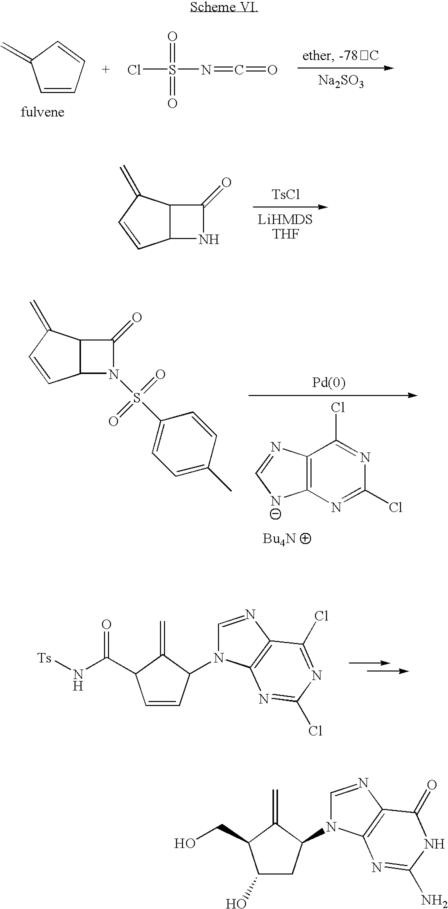

Generally, the synthesis of Entecavir can be achieved by following the reaction steps outlined above for Abacavir, with the exception that fulvene is used as a starting material, rather than cyclopentadiene. A reaction scheme for the synthesis of Entecavir according to the invention is provided in Scheme VI, above.

The present invention being thus described, many variations and modifications may be made to the above-described embodiments. All such modifications and variations are intended to be included herein within the scope of this disclosure and protected by the following claims.

What is claimed is:

1. A process for the preparation of synthetic nucleosides comprising:
a) preparing a bicycloamide derivative of Formula IIa or IIb, 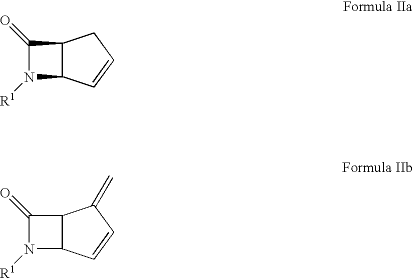
wherein each R¹ is independently an electron withdrawing group;
b) reacting the bicycloamide derivative of Formula IIa or IIb with a nucleic acid base, a heterocyclic base, or salt thereof in the presence of a transition metal catalyst to form a cyclopentenecarboxamide of Formula IVa or IVb; 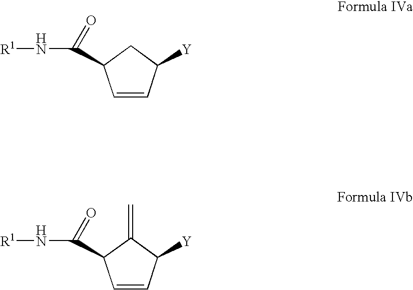
and
c) cleaving a carboxamide group from the cyclopentenecarboxamide to form the synthetic nucleoside.

2. The process of claim 1, wherein the synthetic nucleoside is Abacavir, Carbovir or Entecavir.

3. The process of claim 1, wherein R¹ is selected from the group consisting of benzenesulfonyl chloride, p-toluenesulfonyl chloride, p-methoxybenzenesulfonyl chloride, o-methoxybenzenesulfonyl chloride, p-nitrobenzenesulfonyl chloride, o-chlorobenzenesulfonyl chloride, p-chlorobenzenesulfonyl chloride, p-bromobenzenesulfonyl chloride, p-fluorobenzenesulfonyl chloride, 2,5-dichlorobenzenesulfonyl chloride, methylsulfonyl chloride, camphorsulfonyl chloride, chloroethanesulfonyl chloride, trifluoromethylsulfonyl chloride, and cyclohexanesulfonyl chloride.

4. The process of claim 1, wherein said nucleic acid base is selected from the group consisting of adenine, N⁶-alkylpurines, N6⁶-acylpurines (wherein acyl is C(O)(alkyl, aryl, alkylaryl, or arylalkyl)), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, 5-fluorouracil, C⁵-alkylpyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolo-pyrimidinyl, guanine, adenine, hypoxanthine, 2,6-diaminopurine, and 6-chloropurine.

5. The process of claim 1, wherein said transition metal catalyst is optionally supported and comprises a transition metal selected from the group consisting of Ni, Fe, Co, Pd, Cu, Mo, Ru, Rh, Pt, W, and Ir

6. The process of claim 5, wherein said transition metal catalyst comprises Pd.

7. The process of claim 6, wherein said transition metal catalyst is selected from the group consisting of tetrakis(triphenylphosphine)palladium, tetrakis(triethylphosphine)palladium, tri(dibenzylideneacetone)dipalladium, bis(cycloocta-1,5-dien)palladium, di-μ-chlorobis(η-allyl)dipalladium, palladium acetate, and palladium chloride.

8. The process of claim 1, wherein the synthetic nucleoside is a compound of Formula I: 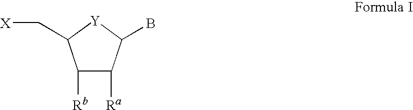
wherein Y is CH₂ or C═CH₂;
B is a purine or pyrimidine base;
X is independently H, OH, alkyl, acyl, phosphate, a lipid, an amino acid, a carbohydrate, a peptide or a cholesterol; and
R^{a} and R^{b} are independently selected from H, OH, alkyl, azido, cyano, alkenyl, alkynyl, Br-vinyl, —C(O)O(alkyl), —O(acyl), —O(alkyl), —O(alkenyl), Cl, Br, F, I, NO₂, NH₂, —NH(alkyl), —NH(cycloalkyl), —NH(acyl), —N(alkyl)₂, —N(acyl)₂; or R^{a} and R^{b} are taken together to form a bond.

9. The process of claim 8, wherein Y is CH₂.

10. The process of claim 9, wherein R^{a} and R^{b} are taken together to form a bond.

11. The process of claim 8, wherein Y is C═CH₂.

12. A process for the preparation of a cyclopentenecarboxamide of Formula IVa or IVb 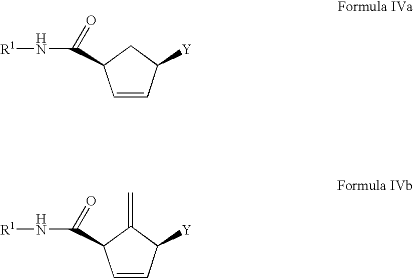
comprising:
a) preparing a bicycloamide derivative of Formula IIa or IIb, 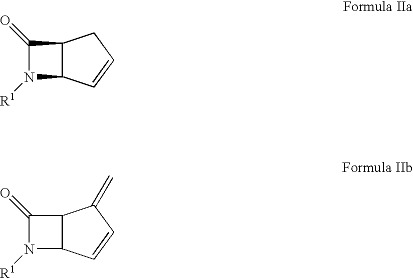
wherein each R¹ is independently an electron withdrawing group;
and
b) reacting the bicycloamide derivative of Formula IIa or IIb with a nucleic acid base or heterocyclic base or salt thereof in the presence of a transition metal catalyst to form a cyclopentenecarboxamide.

13. The process of claim 12, wherein the compound of Formula IVa is selected from the group consisting of: 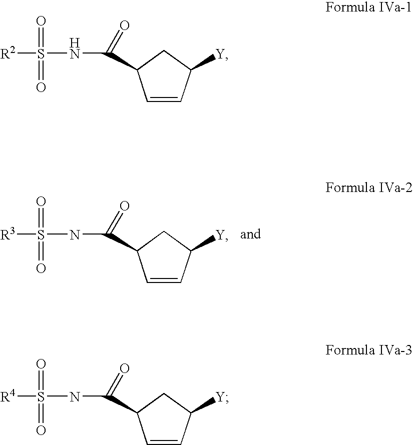
and the compound of Formula IIa is selected from the group consisting of:

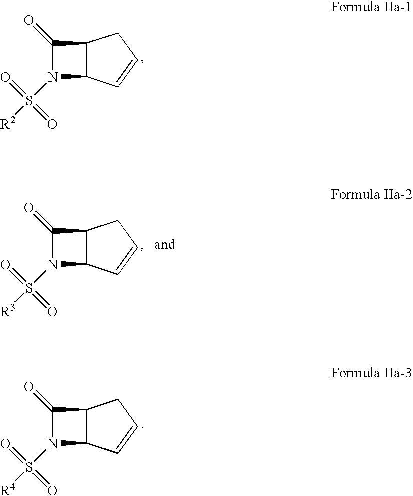

14. The process of claim 12, wherein the compound of Formula IVb is selected from the group consisting of: 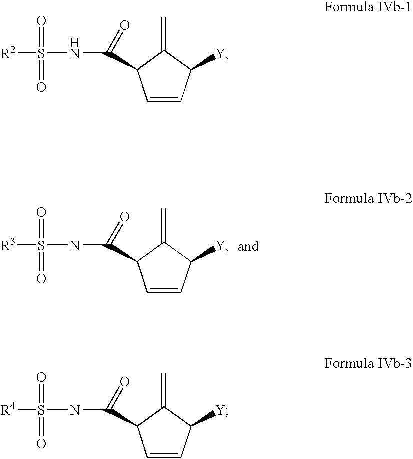
and the compound of Formula IIa is selected from the group consisting of:

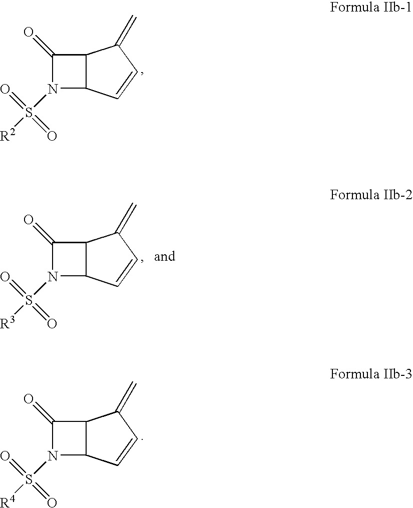

15. The process of claim 12, wherein the transition metal catalyst comprises palladium.

16. The process of claim 15, wherein the transition metal catalyst is selected from the group consisting of: tetrakis(triphenylphosphine)palladium and tetrakis(triethylphosphine)palladium.

17. The process of claim 15, wherein the transition metal catalyst is selected from the group consisting of: tri(dibenzylideneacetone)dipalladium, bis(cycloocta-1,5-dien)palladium, di-μ-chlorobis(η-allyl)dipalladium, palladium acetate, or palladium chloride.

18. The process of claim 17, wherein an organophosphorus compound is added.

19. The process of claim 18, wherein the organophosphorus compound is selected from the group consisting of phosphine, trialkylphosphine, triarylphosphine, triphenylphosphine, tri(o-tolyl)phosphine, trifurylphosphine, bidentate phosphine, Ph₂P(CH₂)ₙPPh₂ where n=2, 3, 4, or 5; phosphite, tri(alkyl)phosphite, tri(aryl)phosphite, tri(ethyl)phosphite, arsine, and triphenylarsine.

20. The process of claim 19, wherein the organophosphorus compound is selected from the group consisting of phosphite, tri(alkyl)phosphite, tri(aryl)phosphite, and tri(ethyl)phosphite.

21. A process for the preparation of a bicycloamide derivative of Formula IIa or IIb 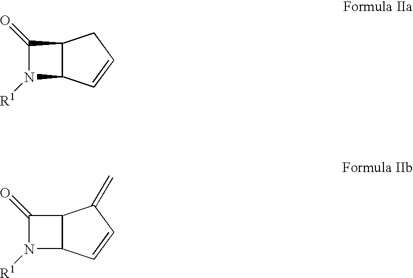
wherein each R¹ is independently an electron withdrawing group;
comprising:
reacting a compound selected from the group consisting of: 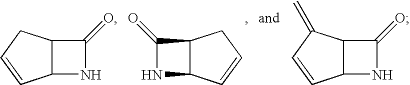
with a compound of formula III,
R¹—X Formula III
wherein X is a halogen.

22. The process of claim 21, wherein the process is conducted in the presence of an organolithium compound.

23. The process of claim 22, wherein the organolithium compound is selected from the group consisting of: alkyl lithium compounds, methyl lithium, n-butyl lithium, t-butyl lithium, aryl lithium compounds, phenyl lithium, lithium amide bases, lithium bis(trimethylsilyl)amide, lithium diisopropylamide, and lithium 2,2,6,6-tetramethyl piperidin-1-ide.

24. The process of claim 21, wherein the reaction is carried out at a temperature of about −78° C. to about 0° C.

25. The process of claim 21, wherein R¹ is an electron withdrawing group that has at least one sulfur, phosphorus or carbon atom which will be bonded to a nitrogen atom of the amide group in the compound of Formula IIa.
